(19) United States
(12) Patent Application Publication
(10) Pub. No.: US 2023/0010442 A1
(43) Pub. Date: Jan. 12, 2023

(54) ABSORBENT ARTICLE WITH REFASTENABLE SIDE SEAMS AND METHOD OF MANUFACTURING

(71) Applicant: Kimberly-Clark Worldwide, Inc., Neenah, WI (US)

(72) Inventors: Danelle M. Buyarski, De Pere, WI (US); Joseph J. Sina, Freedom, WI (US); Cheryl Lancello, Appleton, WI (US); Kathleen I. Bennett, Neenah, WI (US); Janet E. Collins, Hortonville, WI (US)

(21) Appl. No.: 17/757,509

(22) PCT Filed: Dec. 20, 2019
(86) PCT No.: PCT/US2019/067783

§ 371 Date: Jun. 16, 2022

(51) Int. Cl. A61F 13/56; A61F 13/496; A61F 13/493

(52) U.S. Cl. CPC A61F 13/565; A61F 13/496; A61F 13/493

(57) ABSTRACT

Absorbent articles and methods of manufacturing are disclosed. An absorbent article may comprise a chassis comprising a bodyside liner, outer cover, and absorbent body, elastomeric rear side panels and elastomeric front side panels attached to longitudinally extending side edges of the chassis, a portion of each of the elastomeric front and rear side panels and the chassis forming a front waist edge and a rear waist edge, respectively. First and second mechanical fastening components may be coupled to the front waist region and configured to releasably couple to the front waist region to the rear waist region. Each of the rear and front waist edge may further have a width and a ratio of the rear waist edge width to the front waist edge width may be between about 1.5 and about 3.0.

### TECHNICAL FIELD

The present disclosure relates to absorbent articles. More specifically, the present disclosure relates to absorbent articles having refastenable seams.

### BACKGROUND OF THE DISCLOSURE

Garments such as disposable absorbent articles have numerous applications including diapers, training pants, feminine care products, and adult incontinence products. The typical disposable absorbent article is formed as a composite structure including an absorbent structure disposed between a liquid permeable bodyside liner and a liquid impermeable outer cover. These components can be combined with other materials and features such as elastic materials and containment structures to form a product that is specifically suited to its intended purpose.

Prefastened and refastenable absorbent articles can be applied and/or removed either like a conventional diaper or like a conventional training pant. For use as training pants, for example, there may be times when it would be useful to apply the product like a diaper. For instance, it might be more convenient to apply the absorbent article like a diaper when there is a desire not to remove the child's shoes. Because it is difficult to known when a particular mode of applying the absorbent article will be needed, it is beneficial to have an absorbent article that is adaptable to being used either as a diaper or as a pant. This is preferable to keeping both types of absorbent articles available. An absorbent article that can be applied like either a diaper or a pant permits the interior of the absorbent article to be easily checked without having to pull the product downward.

Providing disposable absorbent articles, and particularly prefastened and refastenable absorbent articles, presents many challenges related to ensuring proper fit of the articles on a wearer. As one example, such products incorporate refastenable fasteners which need to be properly aligned and engaged. Many prefastened and refastenable seams of such absorbent articles have typically been placed in the absorbent article such that they will be relatively centered at the side of the wearer of the absorbent article. The location of these refastenable fasteners, which define the refastenable seam in such articles, plays an important role in determining a fit of prefastened and refastenable absorbent articles. There is continued need to provide better fitting prefastened and refastenable absorbent articles and manufacturing techniques for producing such articles.

### SUMMARY OF THE DISCLOSURE

The present disclosure relates to absorbent articles. More specifically, the present disclosure relates to absorbent articles having refastenable seams.

In a first embodiment, an absorbent article with a front waist region having a front waist edge, a rear waist region having a rear waist edge, and a crotch region disposed between the front waist region and the rear waist region may comprise a chassis comprising a bodyside liner, an outer cover, and an absorbent body disposed between the bodyside liner and the outer cover, first and second elastomeric rear side panels, the first elastomeric rear side panel attached to a first longitudinally extending side edge of the chassis and the second elastomeric rear side panel attached to a second longitudinally extending side edge of the chassis, a portion of each of the first and second elastomeric rear side panels and the chassis forming the rear waist edge, first and second elastomeric front side panels, the first elastomeric front side panel attached to the first longitudinally extending side edge of the chassis and the second elastomeric front side panel attached to the second longitudinally extending side edge of the chassis, a portion of each of the first and second elastomeric front side panels and the chassis forming the front waist edge, and first and second mechanical fastening components coupled to the front waist region of the article and configured to releasably couple the front waist region to the rear waist region, wherein each of the rear waist edge and the front waist edge has a laterally extending width, and wherein a ratio of the rear waist edge width to the front waist edge width may be between about 1.5 and about 3.0.

In a second embodiment, an absorbent article with a front waist region having a front waist edge, a rear waist region having a rear waist edge, and a crotch region disposed between the front waist region and the rear waist region and having a body-facing surface and a garment-facing surface may comprise a chassis comprising a bodyside liner, an outer cover, and an absorbent body disposed between the bodyside liner and the outer cover, first and second elastomeric rear side panels, the first elastomeric rear side panel attached to a first longitudinally extending side edge of the chassis and the second elastomeric rear side panel attached to a second longitudinally extending side edge of the chassis, a portion of each of the first and second elastomeric rear side panels and the chassis forming the rear waist edge, first and second elastomeric front side panels, the first elastomeric front side panel attached to the first longitudinally extending side edge of the chassis and the second elastomeric front side panel attached to the second longitudinally extending side edge of the chassis, a portion of each of the first and second elastomeric front side panels and the chassis forming the front waist edge, first and second mechanical fastening components coupled to the front waist region of the article and configured to releasably couple to the front waist region to the rear waist region, and front and rear elastomeric waistbands, each of the front and rear elastomeric waistbands disposed on the garment-facing surface of the article and forming at least part of front waist edge and rear waist edge, respectively, the front elastomeric waistband having a lateral width that is between about 0 mm and about 10 mm less than the front waist edge width, and the rear elastomeric waistband having a lateral width that is between about 0 mm and about 10 mm less than the rear waist edge width. Each of the rear waist edge and the front waist edge has a laterally extending width, and wherein a ratio of the rear waist edge width to the front waist edge width may be greater than about 1.5.

In a third embodiment, an absorbent article with a front waist region having a front waist edge, a rear waist region having a rear waist edge, and a crotch region disposed between the front waist region and the rear waist region and having a body-facing surface and a garment-facing surface may comprise a chassis comprising a bodyside liner, an outer cover, and an absorbent body disposed between the bodyside liner and the outer cover, first and second elastomeric rear side panels, the first elastomeric rear side panel attached to a first longitudinally extending side edge of the chassis and the second elastomeric rear side panel attached to a second longitudinally extending side edge of the chassis, a portion of each of the first and second elastomeric rear side panels and the chassis forming the rear waist edge, first and second elastomeric front side panels, the first elastomeric front side panel attached to the first longitudinally extending side edge of the chassis and the second elastomeric front side panel attached to the second longitudinally extending side edge of the chassis, a portion of each of the first and second elastomeric front side panels and the chassis forming the front waist edge, front and rear elastomeric waistbands, each of the front and rear elastomeric waistbands disposed on the garment-facing surface of the article, the front elastomeric waistband having a lateral width that is between about 0 mm and about 10 mm less than the front waist edge width and the rear elastomeric waistband having a lateral width that is between about 0 mm and about 10 mm less than the rear waist edge width, and first and second mechanical fastening components coupled to the front waist region of the article and configured to releasably couple to the front waist region to the rear waist region, at least a portion of the first and second mechanical fastening components are directly coupled to the front elastomeric waistband. Each of the rear waist edge and the front waist edge has a laterally extending width, and wherein a ratio of the rear waist edge width and the front waist edge width may be greater than about 1.5.

### BRIEF DESCRIPTION OF DRAWINGS

FIG. 1 is a partial schematic view of an absorbent article according to aspects of the present disclosure, where the article is in a wear configuration;

FIG. 2 is a top plan view of absorbent article of FIG. 1 in an unfastened, unfolded, and laid flat condition, and showing the bodyside liner and with portions cut away to show underlying features;

FIG. 3 is a bottom plan view of the absorbent article of FIG. 1 showing the outer cover;

FIG. 4 is a close-up view of a portion of the absorbent article of FIG. 3 showing an exemplary front side panel;

FIG. 4A is a close-up view of a portion of the absorbent article of FIG. 3 showing an alternative front side panel;

FIG. 5 is a close-up view of a portion of the absorbent article of FIG. 3 showing a rear side panel;

FIG. 6 is a cross-section view of the absorbent article of FIG. 2, as viewed along line 6-6;

FIGS. 7A-7C are schematic plan views of an exemplary method for manufacturing absorbent articles according to aspects of the present disclosure; and

FIG. 8 is a top plan view of an absorbent article according to aspects of the present disclosure disposed on a cylinder showing positioning of fastening components.

Repeat use of reference characters in the present specification and drawings is intended to represent the same or analogous features or elements of the disclosure.

### DETAILED DESCRIPTION OF THE DISCLOSURE

The disclosure herein is generally directed towards a prefastened absorbent article and a method of manufacturing such an absorbent article, and which is refastenable by the wearer of the absorbent article. The disclosure herein is generally directed towards prefastened, refastenable articles which have improved fit and comfort for a wearer.

### Definitions

The term “absorbent article” refers herein to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, diaper pants, training pants, youth pants, swim pants, feminine hygiene products, including, but not limited to, menstrual pads or pants, incontinence products, medical garments, surgical pads and bandages, other personal care or health care garments, and the like without departing from the scope of the present disclosure.

The term “bonded” or “coupled” refers herein to the joining, adhering, connecting, attaching, or the like, of two elements. Two elements will be considered bonded or coupled together when they are joined, adhered, connected, attached, or the like, directly to one another or indirectly to one another, such as when each is directly bonded to intermediate elements. The bonding or coupling of one element to another can occur via continuous or intermittent bonds.

The term “cross direction” refers herein to the width of a fabric in a direction generally perpendicular to the direction in which it is produced, as opposed to “machine direction” that refers to the length of a fabric in the direction in which it is produced.

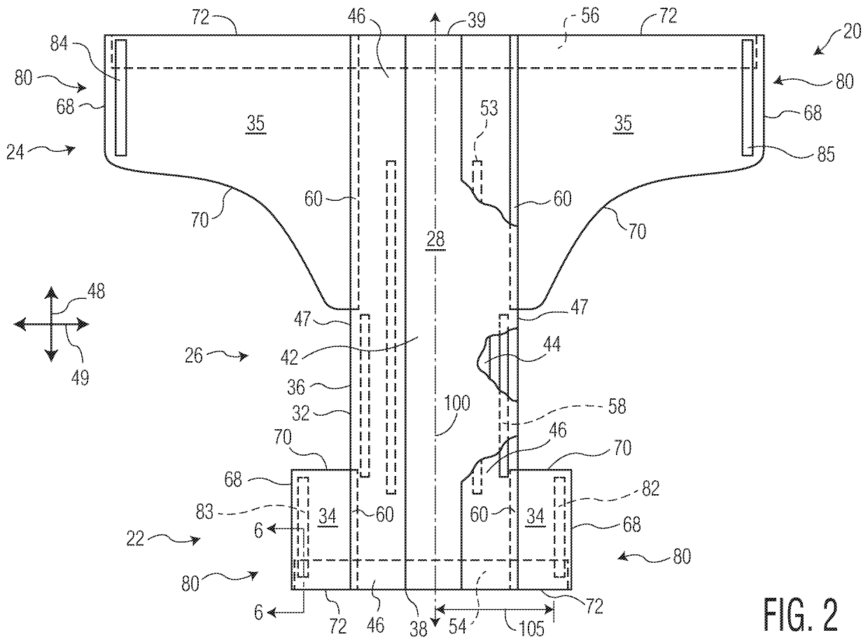

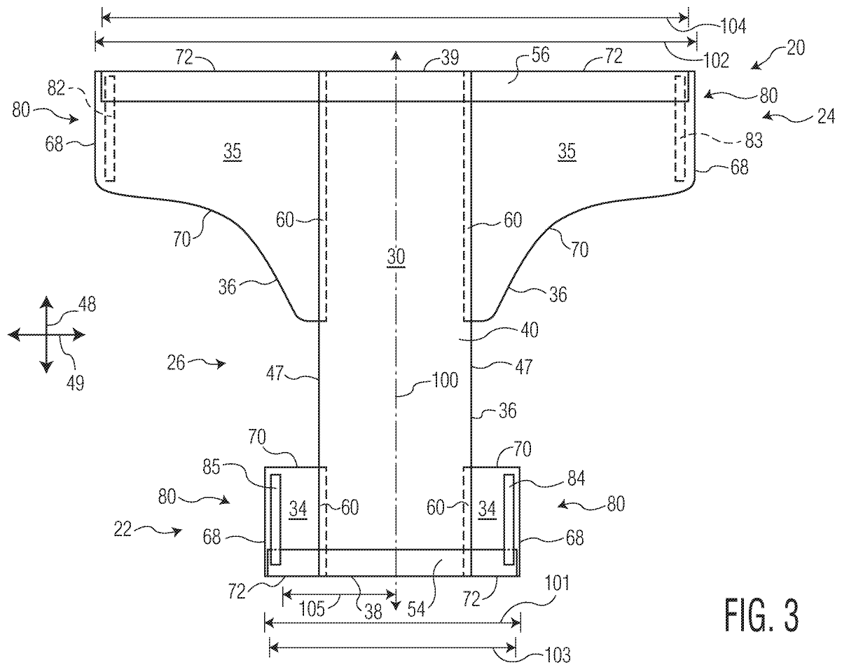

The term “cross direction assembly” refers herein to a process in which disposable absorbent products are manufactured in an orientation in which the products are connected side-to-side, e.g., in the transverse direction shown by arrow 49 in FIGS. 2 and 3. A process utilizing cross direction assembly entails products traveling through a converting machine parallel to the direction of arrow 49, as opposed to “machine direction assembly” in which the products are connected end-to-end or waist-to-waist.

The terms “elastic,” “elasticized” and “elasticity” mean that property of a material or composite by virtue of which it tends to recover its original size and shape after removal of a force causing a deformation.

The term “fabrics” refers herein to any woven, knitted and nonwoven fibrous webs.

The term “film” refers herein to a thermoplastic film made using an extrusion and/or forming process, such as a cast film or blown film extrusion process. The term includes apertured films, slit films, and other porous films that constitute liquid transfer films, as well as films that do not transfer fluids, such as, but not limited to, barrier films, filled films, breathable films, and oriented films.

The term “hydrophilic” refers herein to fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by Cahn SFA-222 Surface Force Analyzer System, or a substantially equivalent system. When measured with this system, fibers having contact angles less than 90 are designated “wettable” or hydrophilic, while fibers having contact angles greater than 90 are designated “nonwettable” or hydrophobic.

The term “liquid impermeable” refers herein to a layer or multi-layer laminate in which liquid body exudates, such as urine, will not pass through the layer or laminate, under ordinary use conditions, in a direction generally perpendicular to the plane of the layer or laminate at the point of liquid contact.

The term “liquid permeable” refers herein to any material that is not liquid impermeable.

The terms “longitudinal” and “transverse” have their customary meaning, as indicated by the longitudinal and transverse axes depicted in FIGS. 2 and 3. The longitudinal axis lies in the plane of the article and is generally parallel to a vertical plane that bisects a standing wearer into left and right body halves when the article is worn. The transverse axis lies in the plane of the article generally perpendicular to the longitudinal axis. The article as illustrated is longer in the longitudinal direction than in the transverse direction.

The term “machine direction” refers herein to the length of a fabric in the direction in which it is produced, as opposed to “cross direction” that refers to the width of a fabric in a direction generally perpendicular to the machine direction.

The term “machine direction assembly” refers herein to a process in which disposable absorbent products are manufactured in an orientation in which the products are connected end-to-end or waist-to-waist, in the longitudinal direction shown by arrow 48 in FIGS. 2 and 3, a process utilizing a machine direction assembly entails products traveling through a converting machine parallel to the direction of arrow 48, as opposed to “cross direction assembly” in which the products are connected side-to-side.

The term “meltblown” refers herein to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which can be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al., which is incorporated herein by reference. Meltblown fibers are microfibers which may be continuous or discontinuous, are generally smaller than about 0.6 denier, and may be tacky and self-bonding when deposited onto a collecting surface.

The term “nonwoven” refers herein to a material which is formed without the aid of a textile weaving or knitting process. The material can have a structure of individual fibers, filaments, or threads (collectively referred to as “fibers”) which can be interlaid, but not in an identifiable manner as in a knitted fabric. Nonwoven material can be formed from many processes such as, but not limited to, meltblowing processes, spunbonding processes, carded web processes, etc.

The term “operatively joined,” in reference to the attachment of an elastic member to another element, means that the elastic member when attached to or connected to the element, or treated with heat or chemicals, by stretching, or the like, gives the element elastic properties; and with reference to the attachment of a non-elastic member to another element, means that the member and element can be attached in any suitable manner that permits or allows them to perform the intended or described function of the joinder. The joining, attaching, connecting or the like can be either directly, such as joining either member directly to an element, or can be indirectly by means of another member disposed between the first member and the first element.

The term “refastenable” refers herein to the property of two elements being capable of releasable attachment, separation, and subsequent releasable reattachment without substantial permanent deformation or rupture.

The terms “releasably attached,” “releasably engaged,” and variations thereof refer herein to two elements being connected or connectable such that the elements tend to remain connected absent a separation force applied to one or both of the elements, and the elements being capable of separation without substantial permanent deformation or rupture. The required separation force is typically beyond that encountered while wearing the absorbent garment. It should be noted that a releasably attached or releasably engaged seam is a refastenable seam that does not include a bonded seam that must be torn, cut, or otherwise disrupted.

The term “spunbond” refers herein to small diameter fibers that are formed by extruding molten thermoplastic material as filaments from a plurality of fine capillaries of a spinnerette having a circular or other configuration, with the diameter of the extruded filaments then being rapidly reduced by a conventional process such as, for example, eductive drawing, and processes that are described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Petersen, and U.S. Pat. No. 3,542,615 to Dobo et al., each of which is incorporated herein in its entirety by reference. Spunbond fibers are generally continuous and often have average deniers larger than about 0.3, and in an embodiment, between about 0.6, 5, and 10 and about 15, 20 and 40. Spunbond fibers are generally not tacky when they are deposited on a collecting surface.

The term “stretchable” refers herein that a material can be stretched, without breaking, to at least 150% of its initial (unstretched) length in at least one direction, suitably to at least 200% of its initial length, desirably to at least 250% of its initial length.

Absorbent Article:

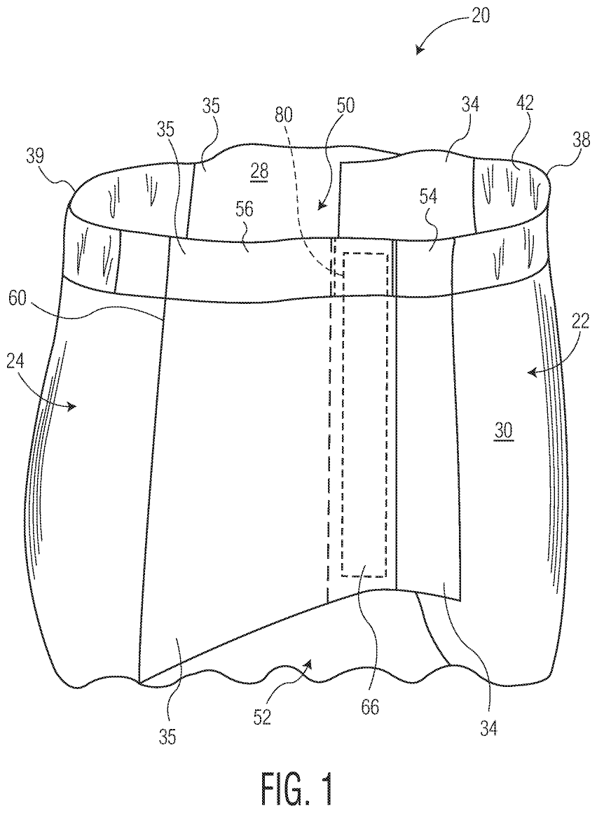

Referring to FIGS. 1-3, an absorbent article 20 is illustrated in the form of a pants-type absorbent article for wear about a wearer's waist. FIGS. 2 and 3 provide illustrations of the absorbent article 20 in an unfastened, unfolded and laid flat configuration with either the outer cover 40 facing the viewer (FIG. 3) or the bodyside liner 42 facing the viewer (FIG. 2). FIG. 1 provides a partial schematic illustration of the absorbent article 20 of FIGS. 2 and 3 in a fully assembled configuration (broadly referred to herein as a wear configuration of the absorbent article 20). The absorbent article 20 can be placed against or in proximity to the body of the wearer to absorb and/or retain various bodily exudates discharged from the body. The absorbent article 20 can be disposable and, therefore, can be discarded after a limited period of use instead of being laundered or otherwise restored for reuse. It is understood that the concepts described herein are suitable for use with various other pants-type absorbent articles such as adult incontinence articles, as well as other articles intended for personal wear such as clothing, diapers, feminine hygiene products, medical garments, surgical pads and bandages, other personal care or health care garments, and the like without departing from the scope of the present disclosure.

The absorbent article 20 can have a longitudinal direction 48 and a transverse direction 49, along with a central longitudinal axis 100. The absorbent article 20 includes a front waist region 22, a rear waist region 24, a crotch region 26 extending longitudinally between and interconnecting the front waist region 22 and rear waist region 24 along a longitudinal direction of the absorbent article 20, an inner surface 28 configured for contiguous relationship with the wearer, and an outer surface 30 opposite the inner surface 28. The absorbent article 20 also has a pair of laterally opposite side edges 36 and a pair of longitudinally opposite waist edges, respectively designated front waist edge 38 and rear waist edge 39 having a front waist edge width 101 and a rear waist edge width 102, respectively. The front waist region 22 is contiguous with the front waist edge 38, and the rear waist region 24 is contiguous with the rear waist edge 39.

The absorbent article 20 includes a central absorbent chassis 32, which when laid flat as in FIGS. 2 and 3 can be rectangular or any other desired shape. A pair of laterally opposite front side panels 34 extends outward from the absorbent chassis 32 at the front waist region 22 (thereby forming transversely outer portions of the front waist region 22, and more broadly in part forming transversely opposite sides of the absorbent article 20). Laterally opposite rear side panels 35 extend outward from the absorbent chassis 32 at the rear waist region 24 (thereby forming transversely outer portions of the rear waist region 24, and together with the front side panels 34 further defining the sides of the absorbent article 20). Where the front side panels 34 and the rear side panels 35 are separate elements bonded to the absorbent chassis 32, the front side panels 34 and the rear side panels 35 can be bonded along seams 60.

As noted previously, the illustrated absorbent article 20 has front side panels 34 and rear side panels 35 defining transversely opposite sides of the absorbent article 20 in the wear configuration of the absorbent article 20. The side panels (34 and 35) can be bonded along seams 60 to the central absorbent chassis 32 in the respective front waist region 22 and rear waist region 24. In various embodiments, the front side panels 34 can be bonded to and extend transversely outward beyond the side edges 47 of the absorbent chassis 32 in the front waist region 22 and the rear side panels 35 can be bonded to and extend transversely outward beyond the side edges 47 of the absorbent chassis 32 in the rear waist region 24. The side panels (34 and 35) can be bonded to the absorbent chassis 32 using bonding means known to those skilled in the art such as adhesive, thermal, pressure or ultrasonic bonding. Alternatively, the side panels (34 and 35) can be formed as an integral portion of a component of the absorbent chassis 32. For example, the side panels (34 and 35) can include a generally wider portion of and be an extension of the outer cover 40, the bodyside liner 42, and/or another component of the absorbent chassis 32.

The front side panels 34 and rear side panels 35 each have an outer edge 68 spaced transversely from the seam 60, a leg end edge 70 disposed toward the longitudinal center of the absorbent article 20, and a waist end edge 72 disposed toward a longitudinal end of the absorbent article 20. The leg end edge 70 and waist end edge 72 extend from the side edges 47 of the absorbent chassis 32 to the outer edges 68. The leg end edges 70 of the side panels (34 and 35) form part of the side edges 36 of the absorbent article 20. In various embodiments, the leg end edges 70 can be curved and/or angled relative to the transverse direction 49 to provide a better fit around the wearer's legs. However, it is understood that only one or both of the leg end edges 70 can be curved or angled, such as the leg end edge 70 of the rear waist region 24, as is shown in FIGS. 2-3, or neither of the leg end edges 70 can be curved or angled, without departing from the scope of this disclosure. The waist end edges 72 are suitably parallel to the transverse direction 49. The waist end edges 72 of the front side panels 34 form part of the front waist edge 38 of the absorbent article 20, and the waist end edges 72 of the rear side panels 35 form part of the rear waist edge 39 of the absorbent article 20. The side panels (34 and 35) suitably, although not necessarily, include a stretchable material capable of stretching in a direction generally parallel to the transverse axis 49 of the absorbent article 20. More suitably the side panels (34 and 35) include an elastic material. Suitable elastic materials, as well as one process of incorporating stretchable side panels into absorbent articles 20, are described in the following U.S. Pat. No. 4,940,464 issued Jul. 10, 1990 to Van Gompel et al.; U.S. Pat. No. 5,224,405 issued Jul. 6, 1993 to Pohjola; U.S. Pat. No. 5,104,116 issued Apr. 14, 1992 to Pohjola; and U.S. Pat. No. 5,046,272 issued Sep. 10, 1991 to Vogt et al.; all of which are incorporated herein by reference. Optionally, the stretch material can include a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. No. 5,226,992 issued Jul. 13, 1993 to Morman; and PCT application WO 01/88245 in the name of Welch et al.; all of which are incorporated herein by reference. In at least some embodiments, side panels (34 and 35) may comprise an elastic film with have cover layers, such as fibrous nonwoven web materials laminated to both sides of the elastic film so as so form a laminate with opposed exterior nonwoven layers and an intermediate layer of elastic film bonded to both of the exterior nonwoven layers. Such exemplary material can be found with within U.S. Pat. No. 7,803,244 to Siqueira et al. and U.S. Pat. No. 8,361,913 to Siqueira et al., each of which is incorporated herein in its entirety by reference. Alternatively, the side panel material can include other woven or nonwoven materials, such as those described above as being suitable for the outer cover 40 or bodyside liner 42; mechanically pre-strained composites; or stretchable but inelastic materials.

While the absorbent article 20 is illustrated as having a pair of front side panels 34 and a pair of rear side panels 35, in various embodiments, the absorbent article 20 can have only a pair of rear side panels 35 which can have a width dimension in the transverse direction 49 such that each of the rear side panels 35 can be fastened to the front waist region 22 of the absorbent article 20 and completely define the sides of the absorbent article 20.

The central absorbent chassis 32 of the absorbent article 20 includes an outer cover 40 and a bodyside liner 42 in a superimposed relationship with the outer cover 40 and bonded to the outer cover 40 by suitable means such as adhesives, ultrasonic bonds, thermal bonds or other conventional techniques. An absorbent structure 44 is disposed between the outer cover 40 and the bodyside liner 42. A pair of containment flaps 46 are bonded to the bodyside liner 42 for inhibiting the transverse flow of body exudates. The central absorbent chassis 32 has opposite ends that form portions of the front and rear waist edges 38 and 39, and opposite side edges 47 that form portions of the side edges 36 of the absorbent article 20.

The outer cover 40 and/or portions thereof can be breathable and/or liquid impermeable. The outer cover 40 can be a single layer of liquid impermeable material, but more suitably includes a multi-layered laminate structure in which at least one of the layers is liquid impermeable. The inner layer of the outer cover 40 can be both liquid and vapor impermeable, or it can be liquid impermeable and vapor permeable. It is also contemplated that the outer cover 40 can be stretchable, and more suitably elastic in at least the transverse direction 49 of the absorbent article 20. In other aspects the outer cover 40 can be stretchable, and more suitably elastic, in both the transverse direction 49 and the longitudinal direction 48. The outer cover 40 may be constructed of a single layer, multiple layers, laminates, spunbond fabrics, films, meltblown fabrics, elastic netting, microporous webs, bonded-carded webs or foams provided by elastomeric or polymeric materials. In an embodiment, for example, the outer cover 40 can be constructed of a microporous polymeric film, such as polyethylene or polypropylene.

In an embodiment, the outer cover 40 can be a single layer of a liquid impermeable material, such as a polymeric film. In an embodiment, the outer cover 40 can be suitably stretchable, and more suitably elastic, in at least the transverse direction 49. In an embodiment, the outer cover 40 can be stretchable, and more suitably elastic, in both the transverse 49 and the longitudinal 48 directions. In an embodiment, the outer cover 40 can be a multi-layered laminate in which at least one of the layers is liquid impermeable. In some embodiments, the outer cover 40 can be a two-layer construction, including an outer layer (not shown) and an inner layer (not shown) which can be bonded together such as by a laminate adhesive. Suitable laminate adhesives can be applied continuously or intermittently as beads, a spray, parallel swirls, or the like, but it is to be understood that the inner layer can be bonded to the outer layer by other bonding methods, including, but not limited to, ultrasonic bonds, thermal bonds, pressure bonds, or the like.

The outer layer of the outer cover 40 can be any suitable material and may be one that provides a generally cloth-like texture or appearance to the wearer. An example of such material can be a 100% polypropylene bonded-carded web with a diamond bond pattern available from Sandler A.G., Germany, such as 30 gsm Sawabond 4185® or equivalent. Another example of material suitable for use as an outer layer of an outer cover 40 can be a 20 gsm spunbond polypropylene non-woven web. The outer layer may also be constructed of the same materials from which the bodyside liner 42 can be constructed as described herein.

The liquid impermeable inner layer of the outer cover 40 (or the liquid impermeable outer cover 40 where the outer cover 40 is of a single-layer construction) can be either vapor permeable (i.e., “breathable”) or vapor impermeable. The liquid impermeable inner layer (or the liquid impermeable outer cover 40 where the outer cover 40 is of a single-layer construction) can be manufactured from a thin plastic film. The liquid impermeable inner layer (or the liquid impermeable outer cover 40 where the outer cover 40 is of a single-layer construction) can inhibit liquid body exudates from leaking out of the absorbent article 20 and wetting articles, such as bed sheets and clothing, as well as the wearer and caregiver.

In some embodiments, where the outer cover 40 is of a single layer construction, it can be embossed and/or matte finished to provide a more cloth-like texture or appearance. The outer cover 40 can permit vapors to escape from the absorbent article 20 while preventing liquids from passing through. A suitable liquid impermeable, vapor permeable material can be composed of a microporous polymer film or a non-woven material which has been coated or otherwise treated to impart a desired level of liquid impermeability.

The absorbent structure 44 can be suitably constructed to be generally compressible, conformable, pliable, non-irritating to the wearer's skin and capable of absorbing and retaining liquid body exudates. The absorbent structure 44 can be manufactured in a wide variety of sizes and shapes (for example, rectangular, trapezoidal, T-shape, I-shape, hourglass shape, etc.) and from a wide variety of materials. The size and the absorbent capacity of the absorbent structure 44 should be compatible with the size of the intended wearer (infants to adults) and the liquid loading imparted by the intended use of the absorbent article 20. The absorbent structure 44 can have a length and width that can be less than or equal to the length and width of the absorbent article 20.

In an embodiment, the absorbent structure 44 can be composed of a web material of hydrophilic fibers, cellulosic fibers (e.g., wood pulp fibers), natural fibers, synthetic fibers, woven or nonwoven sheets, scrim netting or other stabilizing structures, superabsorbent material, binder materials, surfactants, selected hydrophobic and hydrophilic materials, pigments, lotions, odor control agents or the like, as well as combinations thereof. In an embodiment, the absorbent structure 44 can be a matrix of cellulosic fluff and superabsorbent material, or comprise only superabsorbent material in other embodiments. In an embodiment, the absorbent structure 44 may be constructed of a single layer of materials, or in the alternative, may be constructed of two or more layers of materials.

Various types of wettable, hydrophilic fibers can be used in the absorbent structure 44. Examples of suitable fibers include natural fibers, cellulosic fibers, synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers, or composed of nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber. Suitable superabsorbent materials can be selected from natural, synthetic, and modified natural polymers and materials. The superabsorbent materials can be inorganic materials, such as silica gels, or organic compounds, such as cross-linked polymers. In an embodiment, the absorbent structure 44 can be free of superabsorbent material.

If a spacer layer (not shown) is present, the absorbent structure 44 can be disposed on the spacer layer and superposed over the outer cover 40. The spacer layer can be bonded to the outer cover 40, for example, by adhesive. In some embodiments, a spacer layer may not be present and the absorbent structure 44 can directly contact the outer cover 40 and can be directly bonded to the outer cover 40. However, it is to be understood that the absorbent structure 44 may be in contact with, and not bonded with, the outer cover 40 and remain within the scope of this disclosure. In an embodiment, the outer cover 40 can be composed of a single layer and the absorbent structure 44 can be in contact with the singer layer of the outer cover 40. In some embodiments, at least a portion of a layer, such as but not limited to, a fluid transfer layer (not shown) and/or a spacer layer, can be positioned between the absorbent structure 44 and the outer cover 40. The absorbent structure 44 can be bonded to the fluid transfer layer and/or the spacer layer.

The bodyside liner 42 of the absorbent article 20 can overlay the absorbent structure 44 and the outer cover 40 and can isolate the wearer's skin from liquid waste retained by the absorbent structure 44. The bodyside liner 42 can, but need not, have the same dimensions as the outer cover 40. The bodyside liner 42 is suitably compliant, soft feeling, and non-irritating to the wearer's skin.

In various embodiments, a fluid transfer layer can be positioned between the bodyside liner 42 and the absorbent structure 44. In various embodiments, an acquisition layer can be positioned between the bodyside liner 42 and the absorbent structure 44 or a fluid transfer layer, if present. In various embodiments, the bodyside liner 42 can be bonded to the acquisition layer, or to the fluid transfer layer if no acquisition layer is present, via adhesive and/or by a point fusion bonding. The point fusion bonding may be selected from ultrasonic, thermal, pressure bonding, and combinations thereof.

The bodyside liner 42 can be manufactured from a wide selection of materials, such as synthetic fibers (for example, polyester or polypropylene fibers), natural fibers (for example, wood or cotton fibers), a combination of natural and synthetic fibers, porous foams, reticulated foams, apertured plastic films, or the like. Examples of suitable materials include, but are not limited to, rayon, wood, cotton, polyester, polypropylene, polyethylene, nylon, or other heat-bondable fibers, polyolefins, such as, but not limited to, copolymers of polypropylene and polyethylene, linear low-density polyethylene, and aliphatic esters such as polylactic acid, finely perforated film webs, net materials, and the like, as well as combinations thereof.

Various woven and non-woven fabrics can be used for the bodyside liner 42. The bodyside liner 42 can include a woven fabric, a nonwoven fabric, a polymer film, a film-fabric laminate or the like, as well as combinations thereof. Examples of a nonwoven fabric can include spunbond fabric, meltblown fabric, coform fabric, carded web, bonded-carded web, bicomponent spunbond fabric, spunlace, or the like, as well as combinations thereof. The bodyside liner 42 need not be a unitary layer structure, and thus, can include more than one layer of fabrics, films, and/or webs, as well as combinations thereof. For example, the bodyside liner 42 can include a support layer and a projection layer that can be hydroentagled. The projection layer can include hollow projections, such as those disclosed in U.S. Pat. No. 9,474,660 to Kirby, Scott S. C. et al, the entirety of which is hereby incorporated by reference.

For example, the bodyside liner 42 can be composed of a meltblown or spunbond web of polyolefin fibers. Alternatively, the bodyside liner 42 can be a bonded-carded web composed of natural and/or synthetic fibers. The bodyside liner 42 can be composed of a substantially hydrophobic material, and the hydrophobic material can, optionally, be treated with a surfactant or otherwise processed to impart a desired level of wettability and hydrophilicity. The surfactant can be applied by any conventional means, such as spraying, printing, brush coating or the like. The surfactant can be applied to the entire bodyside liner 42 or it can be selectively applied to particular sections of the bodyside liner 42.

In an embodiment, a bodyside liner 42 can be constructed of a non-woven bicomponent web. The non-woven bicomponent web can be a spunbonded bicomponent web, or a bonded-carded bicomponent web. An example of a bicomponent staple fiber includes a polyethylene/polypropylene bicomponent fiber. In this particular bicomponent fiber, the polypropylene forms the core and the polyethylene forms the sheath of the fiber. Fibers having other orientations, such as multi-lobe, side-by-side, end-to-end may be used without departing from the scope of this disclosure. In an embodiment, a bodyside liner 42 can be a spunbond substrate with a basis weight from about 10 or 12 to about 15 or 20 gsm. In an embodiment, a bodyside liner 42 can be a 12 gsm spunbond-meltblown-spunbond substrate having 10% meltblown content applied between the two spunbond layers.

Although the outer cover 40 and bodyside liner 42 can include elastomeric materials, it is contemplated that the outer cover 40 and the bodyside liner 42 can be composed of materials which are generally non-elastomeric. In an embodiment, the bodyside liner 42 can be stretchable, and more suitably elastic. In an embodiment, the bodyside liner 42 can be suitably stretchable and more suitably elastic in at least the lateral or circumferential direction of the absorbent article 20. In other aspects, the bodyside liner 42 can be stretchable, and more suitably elastic, in both the transverse and the longitudinal directions 49, 48, respectively.

The absorbent chassis 32 and side panels (34 and 35) can include two or more separate elements or they can be integrally formed with the absorbent article 20. Integrally formed side panels (34 and 35) and absorbent chassis 32 would include at least some common materials, such as the bodyside liner 42, containment flaps 46, outer cover 40, other materials and/or combinations thereof, and could define a one-piece elastic, stretchable, or nonstretchable absorbent article 20.

The front waist region 22 of the absorbent article 20 can be selectively joined to the rear waist region 24 of the absorbent article 20 via a pair of refastenable side seams 66 to define a pull-on, pant-like (“wear”) configuration of the absorbent article 20 having a waist opening 50 and leg openings 52. Each side seam 66 can be formed from a fastening system 80. The fastening system 80 may include any suitable complementary refastenable fasteners including, for example and without limitation, hook- and loop-type fasteners, other types of mechanical fasteners, adhesive fasteners, cohesive fasteners, and combinations thereof. In some suitable embodiments, the fastening system 80 may be fastened during the manufacturing process of the absorbent article 20 such that the absorbent article 20 is supplied to the wearer in a fastened configuration.

With the absorbent article 20 in the fastened condition as illustrated in FIG. 1, the front side panels 34 and rear side panels 35 are releasably attached to each other by a fastening system 80 to define the assembled three-dimensional wear configuration of the absorbent article 20 having a waist opening 50 and a pair of leg openings 52. The front waist region 22 includes the portion of the absorbent article 20 that, when worn, is positioned at least in part on the front of the wearer while the rear waist region 24 includes the portion of the absorbent article 20 that is positioned at least in part on the rear of the wearer. The crotch region 26 of the absorbent article 20 includes the portion of the absorbent article 20 that is positioned between the legs of the wearer and covers the lower torso of the wearer. In embodiments in which the absorbent article 20 has only a pair of transversely extending rear side panels 35, whether integral or separately attached, a fastener component of the fastening system 80, for example first fastening components 82 and/or 83, can be positioned on the outer cover 40 of the front waist region 22. In such embodiments, an optional complementary fastener could be positioned on the rear side panels 35.

The front side panels 34 and rear side panels 35 include portions of the absorbent article 20 (and more particularly of the front waist region 22 and rear waist region 24) that, when worn, are positioned on the hips of the wearer. The side panels (34 and 35) thus broadly define the transversely opposite sides of the absorbent article 20 at a refastenable seam 66 along which the fastening system 80 releasably attaches the front side panels 34 and rear side panels 35. The waist edges 38 and 39 of the absorbent article 20 are configured to encircle the waist of the wearer and together define the waist opening 50. Portions of the side edges 36 in the crotch region 26 generally define leg openings 52. The side panels (34 and 35) can have a panel length dimension measured parallel to the longitudinal direction 48 along the outer edge 68 of each side panel (34 and 35).

The central absorbent chassis 32 is configured to contain and/or absorb exudates discharged from the wearer. For example, the containment flaps 46 are configured to provide a barrier to the transverse flow of body exudates. A flap elastic member 53 can be operatively joined with each containment flap 46 in any suitable manner as is well known in the art. The elasticized containment flaps 46 define a partially unattached edge that assumes an upright configuration in at least the crotch region 26 of the absorbent article 20 to form a seal against the wearer's body. The containment flaps 46 can be located along the side edges 36 of the absorbent article 20, and can extend longitudinally along the entire length of the absorbent chassis 32 or can only extend partially along the length of the absorbent chassis 32. Suitable constructions and arrangements for the containment flaps 46 are generally well known to those skilled in the art and are described in U.S. Pat. No. 5,895,382 to Robert L. Popp et al. and U.S. Pat. No. 9,259,362 to Robert L. Popp et al, both of which are incorporated herein by reference.

To further enhance containment and/or absorption of body exudates, the absorbent article 20 also suitably includes a front waist elastic member 54, a rear waist elastic member 56, and leg elastic members 58, as are known to those skilled in the art. The leg elastic members 58 can be attached to the outer cover 40 and/or the bodyside liner 42 along the opposite side edges 36 and positioned in the crotch region 26 of the absorbent article 20. The leg elastic members 58 can be longitudinally aligned along each side edge 47 of the absorbent chassis 32. In some embodiments, it may be important for the leg elastic members 58 to not extend into the seam 60 where the side panels (34 and 35) are attached to the absorbent chassis 32. In at least some embodiments, presence of the leg elastic members 58 within a region of the absorbent chassis 32 where the seam 60 is to be formed can interfere with the formation of the bond which creates the seam 60. In some embodiments, the leg elastic members 58 may be applied under tension in a continuous manner, but a portion of the leg elastic members 58 which would overlap the seam 60 are removed, deadened, or otherwise rendered tensionless so as to not interfere with the formation of the seam 60. According to some embodiments, portions of the leg elastic members 58 may overlap the seam 60 for some small portion of the seam 60, but such overlapping portions of the leg elastic members 58 may generally be disposed within the seam 60 in a tensionless configuration.

The waist elastic members (54 and 56) can be attached to the outer cover 40 and/or the bodyside liner 42 along the opposite waist edges (38 and 39) and can extend over part or all of the waist edges (38 and 39). In at least some embodiments as shown in FIGS. 1-5, the waist elastic members (54 and 56) are coupled to the outer surface 30 of the article 20, for example the outer cover 40. Each of the waist elastic members (54 and 56) may be coupled to the article 20 having a front waist elastic member width 103 and a rear waist elastic member width 104. The widths 103, 104 may be wide enough such that the waist elastic members (54 and 56) are coupled to both the absorbent chassis 32 and the front side panels 34, for the front waist elastic member 54, and the rear side panels 35, for the rear waist elastic member 56.

The waist elastic members (54 and 56) may be coupled to the absorbent chassis 32, the front side panels 34, and the rear side panels 35 through any suitable means, for example with adhesives or with mechanical bonds, such as those created through thermal, ultrasonic, or pressure bonding methods common in the industry. In at least some embodiments the waist elastic members (54 and 56) may be coupled to the absorbent chassis 32 proximate the side edges 47 of the chassis 32 with mechanical bonds. The waist elastic members (54 and 56) may further be coupled to the front side panels 34 and the rear side panels 35, respectively, by mechanical bonds proximate outer edges 68 of the side panels (34 and 35). In such embodiments, the waist elastic members (54 and 56) may further be uncoupled to the absorbent chassis 32 and the side panels (34 and 35) between such mechanical bonds. In other embodiments, the side panels (34 and 35) may further be coupled to the chassis 32 and the side panels (34 and 35) by adhesive disposed at relatively low add-on amounts (in gsm).

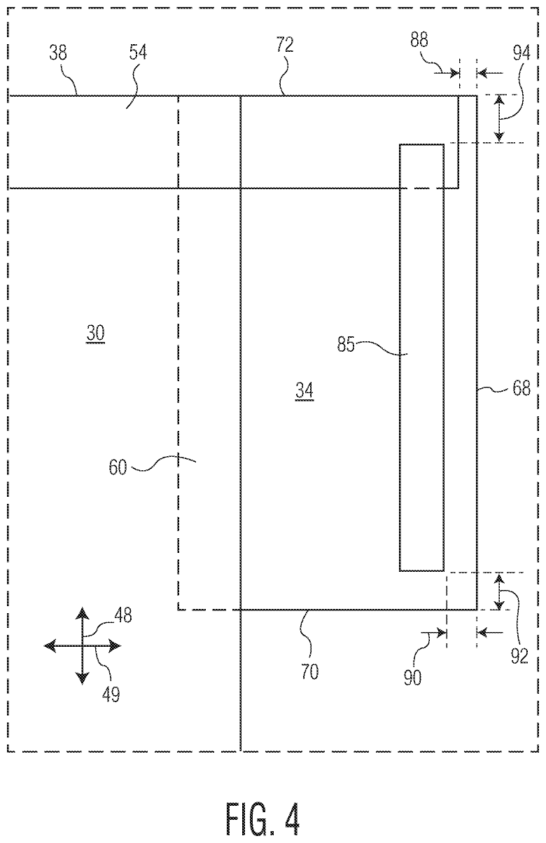

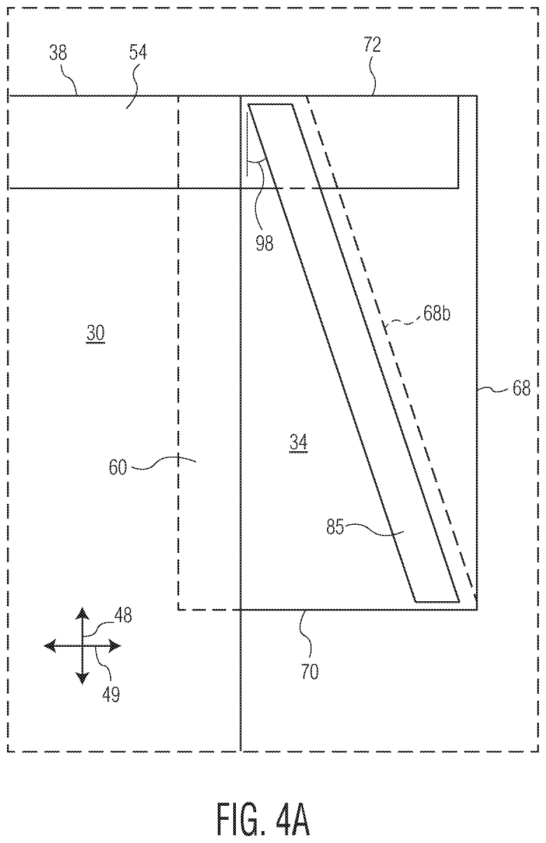

Some preferred embodiments may have the widths 103, 104 be equal to the widths 101, 102. Although in other embodiments, the widths 103, 104 may be between about 0 mm and about 10 mm smaller than the widths 101, 102. In further embodiments, the widths 103, 104 may be between about 0 mm and about 5 mm smaller than the widths 101, 102. In at least some embodiments where the widths 103, 104 are less than the widths 101,102, side edges of the waist elastic members (54 and 56) may be spaced from the side edges 68 of the front and/or rear waist panels 34, 35 a distance 88, as shown in FIG. 4. The distance 88 may generally be between about 0 mm and about 10 mm, or between about 0 mm and about 5 mm. It is also not necessary in all embodiments for the distances 88 on each side of a front or rear waist elastic member (54 or 56) to be equal. That is, the front or rear waist elastic member (54 or 56) could be disposed in a non-centered manner with respect to the absorbent chassis 32 and front and/or rear side panels 34, 35. In such embodiments, when the article 20 is in a wear configuration, the waist elastic members (54 and 56) can form a generally fully-encircling waist elastic member, which may provide a variety of benefits to the article 20—for example from a fit perspective and/or from a perception perspective.

It should be understood that it is not necessary that the widths 103, 104 have the same relation to the widths 101, 102. For example, only one of the widths 103, 104 could be equal to the width 101, 102, or the widths 103, 104 could be different from the widths 101, 102 by different amounts.

The waist elastic members (54 and 56) may be applied to the article 20 such that the waist elastic members (54 and 56) generally form a part of the front and rear waist edges (38 and 39) respectively. For instance, the waist elastic members (54 and 56) may each have an upper edge which generally aligns with edges of the outer cover 40 in the front and/or rear waist regions 22, 24. Combined, these edges of the waist elastic members (54 and 56) and outer cover 40 form the front and rear waist edges (38 and 39). In other embodiments, however, edges of the waist elastic members (54 and 56) may not be aligned with the front and rear waist edges (38 and 39).

The front and/or rear waist elastic members (54 and/or 56) may further be applied to the absorbent chassis 32 and front and rear side panels (34 and 35) while under tension. In some embodiments, the front and/or rear waist elastic members (54 and/or 56) may be applied to the absorbent chassis 32 and front and rear side panels (34 and 35) at greater than about 100% extension and less than about 130% extension, or between about 105% extension and about 130% extension, or between about 110% extension and about 130% extension. As used herein, percent extension is a value that defines a length of a material as a percent of its unstretched length. Accordingly, a material having a 100% extension represents the length of the material in its fully-laid out, but unstretched state, while 110% extension represents the material stretched by 10% to have a stretched length that is 110% of its unstretched length.

It should be further understood that the front and/or rear waist elastic members (54 and/or 56) could be applied at different extensions. For example, in some preferred embodiments, the front waist elastic member 54 may be applied in a latent state (e.g. 100% extension, or less than 100% extension) while the rear waist elastic member 56 is applied at a greater than 100% extensions, or greater than 110% extension, or greater than 120% extension. In other embodiments, the front waist elastic member 54 may be applied to the absorbent chassis 32 and front and rear side panels (34 and 35) at an extension of between about 100% and about 105% while the rear waist elastic member 56 is applied at an extension of between about 110% and about 130%. In general, the rear waist elastic member 56 may be applied at a percent extension that is greater than the percent extension at which the front waist elastic member 54 is applied.

In some particularly preferred embodiments where the article 20 has both front and rear side panels (34 and 35), it has been found that it is desirable for the rear waist elastic member 56 to be applied at between about 115% and about 125% extension while the front waist elastic member 54 is also applied between about 115% and about 125% extension. In at least some of these embodiments, the applied extension of the waist elastic members (54 and 56) is the same. In other embodiments, the applied extension of the waist elastic members (54 and 56) is different, but a difference between the applied extensions of the waist elastic members (54 and 56) is between about 0% and about 5%. It has been found that these described applied extensions help to result in an article 20 having a desired un-tensioned waist circumference in a wear configuration for the various front and rear waist edge width 101, 102 dimensions disclosed herein.

In other preferred embodiments where the article 20 has front and rear side panels (34 and 35), it has been found that where the rear waist elastic member 56 is applied at greater than about 125% extension, the front waist elastic member 54 should be applied at less than about 105% extension. In further embodiments, where the rear waist elastic member 56 is applied at greater than about 125% extension and less than about 135% extension, the front waist elastic member 54 should be applied at greater than or equal to about 100% extension and less than about 105% extension. It has been found that these described applied extensions help to result in an article 20 having a desired un-tensioned waist circumference in a wear configuration for the various front and rear waist edge width 101, 102 dimensions disclosed herein.

Where the article 20 has only rear side panels 35, it has been found that it is desirable for the rear waist elastic member 56 to be applied at between about 110% and about 120% extension while the front waist elastic member 54 is also applied between about 110% and about 120% extension. In at least some of these embodiments, the applied extension of the waist elastic members (54 and 56) is the same. In other embodiments, the applied extension of the waist elastic members (54 and 56) is different, but a difference between the applied extensions of the waist elastic members (54 and 56) is between about 0% and about 5%. It has been found that these described applied extensions help to result in an article 20 having a desired un-tensioned waist circumference in a wear configuration for the various front and rear waist edge width 101, 102 dimensions disclosed herein.

In other preferred embodiments where the article 20 has only rear side panels 35, it has been found that where the front waist elastic member 54 is applied at less than about 105% extension, the rear waist elastic member 56 should be applied at greater than about 115% extension. In further embodiments, where the front waist elastic member 54 is applied at greater than or equal to about 100% and less than about 105% extension, the rear waist elastic member 56 should be applied at greater than about 115% extension and less than about 125% extension. It has been found that these described applied extensions help to result in an article 20 having a desired un-tensioned waist circumference in a wear configuration for the various front and rear waist edge width 101, 102 dimensions disclosed herein.

In may be beneficial for the front waist elastic member 54 and/or the rear waist elastic member 56 to have stretch properties that are similar to the front and/or rear waist side panels 34, 35. In some preferred embodiments, the front and/or rear waist side panels 34, 35 may be extensible up to between about 180% to about 250% of their unstretched length (sometimes referred to as a stretch-to-stop extension value). Accordingly, the front and/or rear waist elastic members (54 and/or 56) may be extensible within this range as well.

Additionally, it is not necessary in all embodiments for the extensibility of the front and/or rear waist elastic members (54 and/or 56) to match the extensibility of the front and/or rear waist side panels (34 and 35). Instead, the extensibilities may be different, but the extensibilities of the front and/or rear waist elastic members (54 and/or 56) and the front and/or rear waist side panels (34 and 35) may both be within the range of about 180% to about 250% extensibility. One way to achieve such different elastic properties for the front and rear waist elastic members (54 and 56) would be to use different materials for the front waist elastic member 54 and the rear waist elastic member 56.

It has been found that for articles 20 having both front and rear side panels (34 and 35), it may be beneficial for the extensibility of the front and rear side panels (34 and 35) to differ. In any of the embodiments described herein where the article 20 has both front and rear side panels (34 and 35), it may be beneficial for the rear side panels 35 to have an extensibility of between about 170% and about 190%, or between about 175% and about 185%. In such embodiments, the front side panels 34 may have an extensibility of between about 110% and about 130%, or between about 115% and about 125%. These values may help to ensure that resulting article 20 has a desired un-tensioned waist circumference in a wear configuration for the various front and rear waist edge width 101, 102 dimensions disclosed herein.

For articles 20 having only rear side panels 35, it has been found that the extensibility of the rear side panels 35 should be between about 170% and about 190%. In more specific embodiments, the extensibility of the rear side panels 35 should be between about 175% and about 185%. These values may help to ensure that resulting article 20 has a desired un-tensioned waist circumference in a wear configuration for the various front and rear waist edge width 101, 102 dimensions disclosed herein.

The absorbent article 20 includes a fastening system 80 for refastenably securing the absorbent article 20 about the waist of the wearer and forming refastenable seams 66 when the article 20 is in a wear configuration. The fastening system 80 includes first fastening components (82 and 83) that are adapted to refastenably connect to mating second fastening components (84 and 85). In an embodiment, one surface of each of the first fastening components (82 and 83) can have a plurality of engaging elements that project from that surface. The engaging elements of the first fastening components (82 and 83) are adapted to repeatedly engage and disengage engaging elements of the second fastening components (84 and 85).

In an embodiment, the first fastening components (82 and 83) each have hook type fasteners and the second fastening components (84 and 85) each have complementary loop type fasteners. In another embodiment, the first fastening components (82 and 83) each have loop type fasteners and the second fastening components (84 and 85) each have complementary hook type fasteners. Alternatively, the fastening components 82, 83, 84, 85 can be interlocking similar surface fasteners; adhesive or cohesive fastening elements such as an adhesive fastener and an adhesive-receptive landing zone or material; or the like.

In further embodiments, the first or second fastening components 82, 83 or 84, 85 may comprise the material forming the side panels (34 or 35). For example, in these embodiments there may be no separate complementary loop type fasteners coupled to the side panels (34 or 35). Instead, the material forming the side panels (34 or 35) itself is the other of the first or second fastening components 82, 83 or 84, 85 and is configured to be releasably engageable with the first or second fastening components 82, 83 or 84, 85 which comprise the hook type fasteners.

Where the second fastening components (84 and 85) comprises separate loop elements attached to the rear side panels 35, the second fastening components (84 and 85) can be positioned on the inner facing surface 28 of the absorbent article 20 in the rear waist region 24. The second fastening components (84 and 85) are sized to receive the first fastening components (82 and 83) and can be positioned along the outer edges 68 of the rear side panels 35. The second fastening components (84 and 85) may be positioned on on the rear side panels 35 in relation to the waist end edge 72 and the leg end edge 70 in a similar fashion as the first fastening components (82 and 83) are disposed in relation to the edges 72, 70 of the front side panels 34.

Loop type fasteners can be a fabric or material having a base or rearing structure and a plurality of loop members extending upwardly from at least one surface of the rearing structure. The loop material can be formed of any suitable material, such as acrylic, nylon, polypropylene or polyester, and can be formed by methods such as warp knitting, stitch bonding, or needle punching. Loop type materials can also comprise any fibrous structure capable of entangling or catching hook type materials, such as carded, spunbond, or other nonwoven webs or composites, including elastomeric and nonelastomeric composites.

Hook type fasteners can be a fabric or material having a base or rearing structure and a plurality of hook members extending upwardly from at least one surface of the rearing structure. In contrast to the loop type fasteners which can be flexible fabric, the hook material can be a resilient material to minimize unintentional disengagement of the fastener component as a result of the hook material becoming deformed and catching on clothing or other items. The term “resilient” refers to an interlocking material having a predetermined shape and the property of the interlocking material to resume the predetermined shape after being engaged and disengaged from a mating, complementary interlocking material. Suitable hook material can be molded or extruded of nylon, polypropylene, or another suitable material. Suitable hook materials area available from commercial vendors such as Velcro Industries and Minnesota Mining & Manufacturing Co.

The first fastening components (82 and 83) can be positioned at the outer facing surface 30 of the absorbent article 20 in the front waist region 22. For example, the first fastening components (82 and 83) can be positioned proximate the outer edges 68 of the front side panels 34. Although, in embodiments where the article 20 does not have front side panels, the first fastening components (82 and 83) can be positioned proximate the side edges 47 of the absorbent chassis 32 on the outer surface 30.

Where the article 20 comprises front side panels 34, as seen more clearly in FIG. 4, the first fastening components (82 and 83) may be disposed a distance 90 from the outer edges 68 of the front side panels 34. The distance 90 may generally be between about 0 mm and about 10 mm, or between about 5 mm and about 10 mm. The first fastening components (82 and 83) may be spaced from the leg end edge 70 of the front side panels 34 a distance 92. The distance 92 may generally be between about 5 mm and about 30 mm, or between about 5 mm and about 20 mm. The first fastening components (82 and 83) may further be spaced from the waist end edge 72 of the front side panels 34 a distance 94. The distance 92 may generally be between about 5 mm and about 30 mm, or between about 5 mm and about 20 mm. In general, it is preferred that the first fastening components (82 and 83) span a majority of the length of the front side panels 34 between the leg end edge 70 and the waist end edge 72, for example between about 80 to about 100 percent, or between about 90 to 100 percent, or between about 98 to about 100 percent of the distance between the waist opening 50 and the leg openings 52, which distance is measured parallel to the longitudinal direction 48. In general, some small spacing between the first fastening components (82 and 83) and the edges 70, 72 may be preferred in order to prevent the first fastening components (82 and 83) from rubbing on the skin of a wearer and to create softer edges (e.g. edges 68, 70, and/or 72) of the front side panels 34. The first fastening components (82 and 83) themselves may have widths of between about 5 mm and about 15 mm, or more preferably between about 7 mm and about 13 mm.

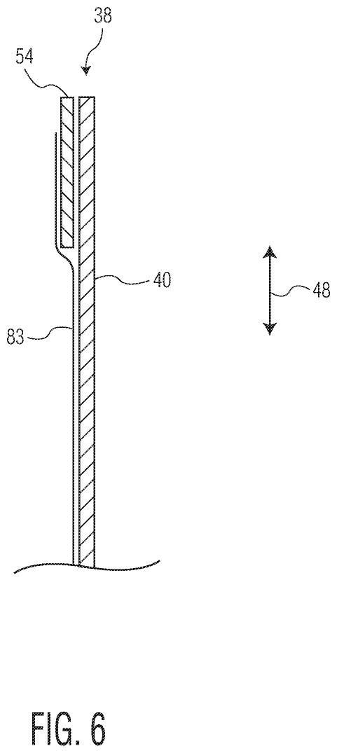

The first fastening components (82 and 83) are additionally disposed on top of the front waist elastic member 54, as can be seen more clearly in FIG. 6. In these configurations, at least a portion of each of the first fastening components (82 and 83) are disposed so as to attach to the rear side panels 35 such that a portion of the rear waist elastic member 56 covers portions of the first fastening components (82 and 83), albeit with the rear side panels 35 disposed between the first fastening components (82 and 83) and the rear waist elastic member 56. In this manner, the front and rear waist elastic members 54, 56 may be coupled together to form a fully encircling waist elastic member.

As shown in FIG. 4, the first fastening components (82 and 83) may generally extend in a parallel manner with respect to the central longitudinal axis 100 of the article 20. Although, as shown in FIG. 4A, some embodiments of the present disclosure contemplate the first fastening components (82 and 83) forming angles 98 with respect to the central longitudinal axis 100. The angles 98 may be preferred to be between about 0 degrees and about 45 degrees, or between about 0 degrees and about 35 degrees, or between about 0 degrees and about 30 degrees, or between about 5 degrees and about 30 degrees, or between about 10 degrees and about 30 degrees, or between about 20 degrees and about 30 degrees. Such values for the angles 98 may provide the article 20 with superior fit characteristics, since the refastenable seam 66 may extend along a wearer's waist and hip flexor region in a manner where the seam 66 is subjected to less bending and twisting forces while the wearer moves.

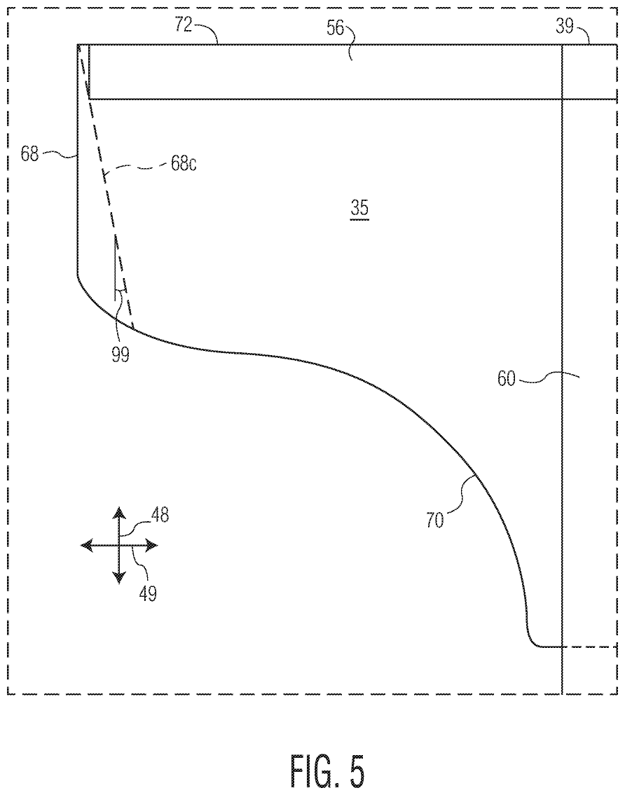

In at least some of these embodiments, outer edges 68 of the elastomeric front side panels 34 may also form an angle with respect to the central longitudinal axis 100, as shown by optional dashed outer edge 68b in FIG. 4A. In these embodiments, the angle formed by the elastomeric front side panels 34 with respect to the central longitudinal axis 100 may equal the angle 98. In other embodiments, the angle formed by the elastomeric front side panels 34 with respect to the central longitudinal axis 100 may be within about 10 degrees of the angle 98, or within about 5 degrees of the angle 98. Further embodiments may have outer edges 68 of the elastomeric rear side panels 35 forming an angle 99 with respect to the central longitudinal axis 100, as shown by optional outer edge 68c in FIG. 5. The angle 99 may be equal to the angle 98 in some embodiments, while in other embodiments, the angle 99 may be within about 10 degrees of the angle 98, or within about 5 degrees of the angle 98.

In various embodiments, it may be preferable that the refastenable side seams 66 are positioned closer to the front of the wearer rather than the rear or the sides of the wearer. The side panels (34 and 35), combined with the central absorbent chassis 32, form the front and rear waist edges 38, 39 having front and rear waist edge widths 101, 102. In order to position the seams 66 closer to the front of the wearer, the rear waist edge width 102 may generally be greater than the front waist edge width 101.

More specifically, it has been found that it is desirable for the refastenable seam 66 to be disposed within a narrow region around the circumference of the waist of a wearer in order to provide a desired fit of the article 20 and limit or prevent certain deficiencies of the article 20 when worn. For example, when located in the desired region around the circumference of the waist of a wearer, the refastenable seams 66 are located away from the hip flexor of the wearer. This helps to prevent the first fastening components (82 and 83) from bending or twisting, which can lead to de-coupling of the refastenable seam 66 and/or irritation for the wearer, as the first fastening components (82 and 83) tend to be more stiff than the elastomeric side panels.

However, it is also desirable that the refastenable seams 66 not be located to close to the central longitudinal axis 100 of the article 20. For example, where the refastenable seams 66 are located a distance 105 from the central longitudinal axis 100 that is less than about 15 mm, the rear side panels 35 significantly overlap the absorbent chassis 32 in the front waist region 22. Such high-degree of overlap between the side panels 35 and the absorbent chassis 32 results in covering up any printed graphics in the front waist region 22 of the chassis 32, which is desirable feature in many absorbent articles. Where the refastenable seams 66 disposed further away from the central longitudinal axis 100 so as to allow room for printed graphics on the front waist region 22 to be seen by a wearer, the refastenable seams 66 may still be located too close to the central longitudinal axis 100 where the seams 66 are located a distance 105 of between about 15 mm and about 40 mm from the central longitudinal axis 100. For example, where the refastenable seams 66 are located within this range, the seams 66 may be located at locations around the circumference of a waist of a wearer where the stresses and strains of movement by the wearer's hip flexor are the greatest. These stresses and strains can cause detachment of the refastenable seams 66 and/or irritation to the wearer due to the bending and flexing of the first fastening components (82 and 83) which can comprise relatively stiff materials in some embodiments.

Additionally, it may be preferable for the first fastening components (82 and 83) to be located on the elastomeric front side panels 34 as opposed to on the absorbent chassis 32 to aid in comfort of the wearer. With this problem in mind, it has been found that to effectively locate the refastenable seams 66 within a desired region on the waist of a wearer, particular dimensions and ratios of the rear waist edge width 102 to the front waist edge width 101 should be used.

According to some embodiments of the present disclosure, a preferred ratio of the rear waist edge width 102 to the front waist edge width 101 is between about 1.25 and about 3.0, or more specifically between about 1.5 and about 3.0, or more specifically between about 1.5 and about 2.75, or more specifically between about 1.5 and about 2.5, or more specifically between about 1.5 and about 2.25, or more specifically between about 1.5 and about 2.0, or more specifically between about 1.6 and about 1.8.

It has been found that some particularly advantageous example articles 20 include front side panels 34 and have a front waist edge width 101 of between about 175 mm and about 225 mm. In order to locate the refastenable seams 66 within the desired range around the circumference of the waist of a wearer, the rear waist edge width 102 of such articles 20 should be between about 300 mm and about 400 mm, or more specifically between about 300 mm and about 375 mm, or more specifically between about 315 mm and about 375 mm, or more specifically between about 315 mm and about 360 mm.

In further advantageous embodiments, articles 20 according to the present disclosure may not have front side panels 34. In such embodiments, the articles 20 may have a front waist edge width 101, which is thus equal to a width of the absorbent chassis 32, of between about 120 mm and about 160 mm. In order to locate the refastenable seam 66 within the desired region around the circumference of the waist of a wearer, the rear waist edge width 102 should be between about 325 mm and about 425 mm, or more specifically between about 350 mm and about 425 mm, or more specifically between about 350 mm and about 400 mm.

In further embodiments, it may be preferable to have the front fastening elements (82 and 83) located in particular locations with respect to the central longitudinal axis 100 of the article 20, to assist in locating the refastenable seams 66 within the desired region around the circumference of the waist of a wearer. As seen most clearly in FIG. 2, inboard edges of the first fastening components (82 and 83) are disposed a distance 105 from the central longitudinal axis 100. It has been found that useful distances 105 for ensuring desired placement of the seams 66 around the circumference of the waist of a wearer are between about 45 mm and about 90 mm, or between about 60 mm and about 90 mm, or between about 60 mm and about 85 mm, or between about 60 mm and about 80 mm, or between about 70 mm and about 80 mm. Where the first fastening components (82 and 83) are disposed at an angle with respect to the central longitudinal axis 100, as in FIG. 4A, the distance 105 may be measured from an edge of first fastening components (82 and 83) most proximate the front waist edge 38.

In some embodiments where the article 20 includes both front and rear side panels (34 and 35), the front waist edge width 101 may be between about 180 mm and about 220 mm and the rear waist edge width 102 may be between about 300 mm and about 330 mm. In such embodiments, the distance 105 may be between about 45 mm and about 90 mm. In other of these embodiments, the front waist edge width 101 may be between about 180 mm and about 220 mm and the rear waist edge width 102 may be between about 325 mm and about 355 mm. In these particular embodiments, the distance 105 may be between about 45 mm and about 90 mm. In still further preferred embodiments, which include both front and rear side panels (34 and 35), the front waist edge width 101 may be between about 180 mm and about 220 mm and the rear waist edge width 102 may be between about 345 mm and about 375 mm. In such embodiments, the distance 105 may be between about 45 mm and about 90 mm. In any of the above embodiments of this paragraph, the front waist edge width 101 may be more preferred to be between about 185 mm and about 215 mm, or between about 190 mm and about 210 mm. In any of the above embodiments of this paragraph, the distance 105 may be between about 65 mm and about 80 mm.

In some embodiments where the article 20 only includes rear side panels 35, the front waist edge width 101 may be between about 120 mm and about 160 mm and the rear waist edge width 102 may be between about 335 mm and about 365 mm. In such embodiments, the distance 105 may be between about 45 mm and about 60 mm. In other of these embodiments, the front waist edge width 101 may be between about 120 mm and about 160 mm and the rear waist edge width 102 may be between about 365 mm and about 395 mm. In such embodiments, the distance 105 may be between about 45 mm and about 60 mm. In still further preferred embodiments, which include only the rear side panels 35, the front waist edge width 101 may be between about 120 mm and about 160 mm and the rear waist edge width 102 may be between about 385 mm and about 415 mm. In such embodiments, the distance 105 may be between about 45 mm and about 60 mm. In any of the above embodiments of this paragraph, the front waist edge width 101 may be more preferred to be between about 125 mm and about 155 mm, or between about 130 mm and about 150 mm. In any of the above embodiments of this paragraph, the distance 105 may be between about 50 mm and about 60 mm.

In still further embodiments, the article 20 may be constructed so as to position the refastenable seams 66 within a desired range around the circumference of a waist of a wearer. A Seam Position Test Method can be used to determine whether the refastenable seams 66 are located at such a desired location. The Seam Position Test Method comprises placing an absorbent article with the seams 66 in a fastened state on a cylinder and measuring the location of the fastening components forming the seams 66.

The cylinder should have an outer circumference of 500 mm. The cylinder should be made of a rigid material that is resistant to pressure applied to its outer surface. The absorbent article is preferably a new article that is pulled from a fresh package of absorbent articles. The longitudinal centerline of the article should be marked with a suitable marking device, such as a pen or marker.

Where the seams 66 are in a fastened state in a new article, the seams 66 should be maintained in this fastened state which was done by the article manufacturer. Where the seams 66 are unfastened, the seams 66 should be fastened so as connect first fastening components to second fastening components of the article. Where the first fastening components and the second fastening components have different widths, the seams 66 should be formed so that the rear panels overlap the front panels of the article for a distance of 10 mm beyond an inner edge of the fastening components. Where the article comprises only first fastening components and where the first fastening components are disposed on the front panels, the seams 66 should be fastened so that the rear panels overlap the front panels for a distance of 10 mm beyond inner edges of the first fastening components. Where the article comprises only first fastening components and where the first fastening components are disposed on the rear panels, the seams 66 should be fastened so that the front panels overlap the rear panels for a distance of 10 mm beyond inner edges of the first fastening components.

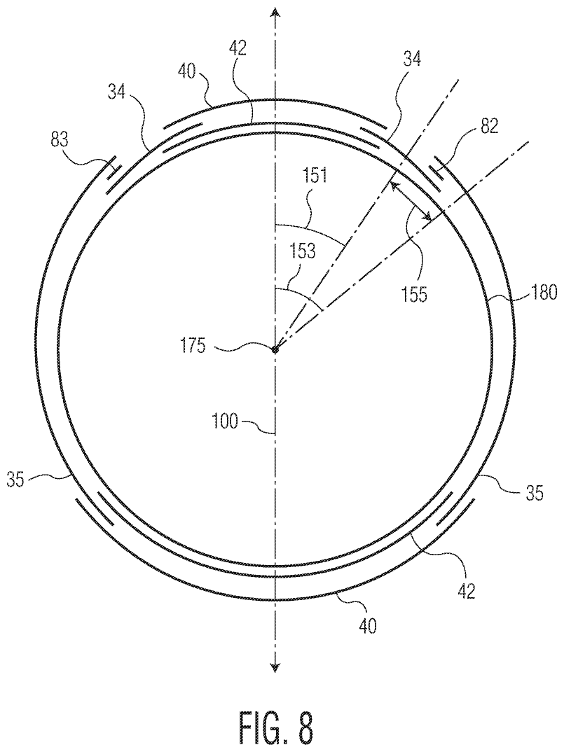

Once the article has been marked and placed into a fastened state, the article should be placed onto the cylinder with the cylinder extending through the waist opening, as shown in FIG. 8 with cylinder 180. In this state, the location of the inner edges of the fastening components forming the seams 66 around the circumference of the cylinder 180 may be determined. More specifically, where the fastening components comprise hook components, inner edges of the hook components should be identified. Where the fastening components do not comprise hook components, inner edges of the fastening components coupled to the front panels should be identified. Where the fastening components do not comprise hook components and the front panels do not comprise fastening components, inner edges of the fastening components coupled to the rear panels should be identified.

Next, an angle formed by a line drawn between the identified inner edge of the fastening components and the longitudinal centerline 100 with both lines extending through the center point 175 of the cylinder 180 should be determined. In FIG. 8, angles 151 and 153 are shown which help to define region 155 on the outer circumference of the cylinder 180, region 155 being the desired region in which the seams 66 are located. It has been found that it is most preferable for the angle formed by a line drawn between the identified inner edge of the fastening components and the longitudinal centerline 100 to be between about 50 degrees and about 75 degrees, or more preferably between about 55 degrees and about 70 degrees, or more preferably between about 50 degrees and about 65 degrees, as determined according to the Seam Position Test Method. This positioning translates to a desired positioning of the seams 66 on the circumference of a wearer to achieve the benefits described herein. Current refastenable absorbent articles tend to place the seams 66 such that an angle determined by the Seam Position Test Method is between about 80 degrees and about 100 degrees.

The various components of the absorbent article 20 can be bonded together by any means known to those skilled in the art such as, for example, adhesive, thermal and/or ultrasonic bonds. Suitable absorbent supply mechanisms, web unwinds, conveyor systems, registration systems, drive systems, control systems, and the like, for use in the manufacture of the absorbent article 20 are disclosed in U.S. Pat. No. 5,766,389 to Brandon et al. which is incorporated herein by reference to the extent it does not conflict with the disclosure herein. Many manufacturing processes to make an absorbent article 20 generally produce a continuous web of interconnected absorbent articles 20.

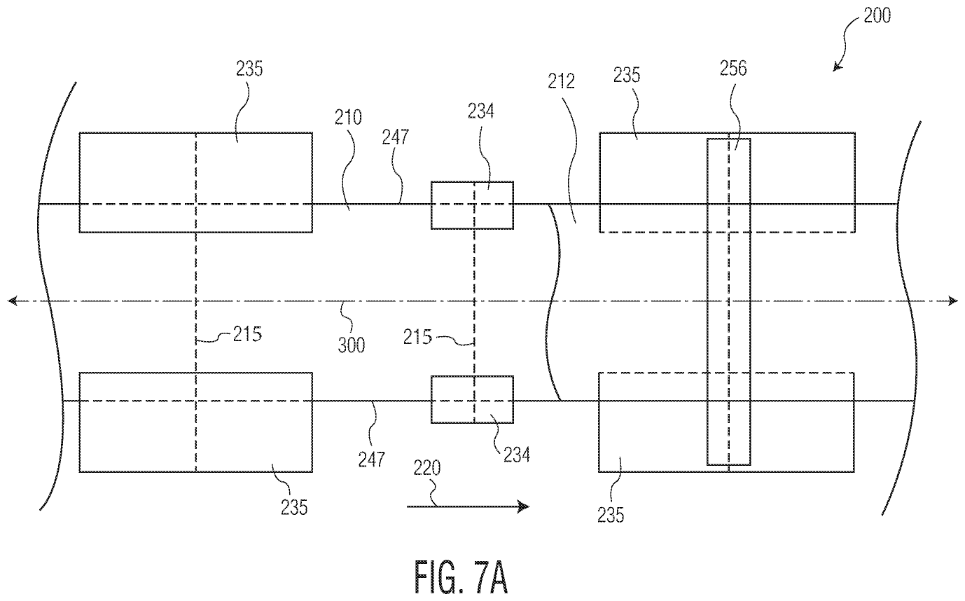

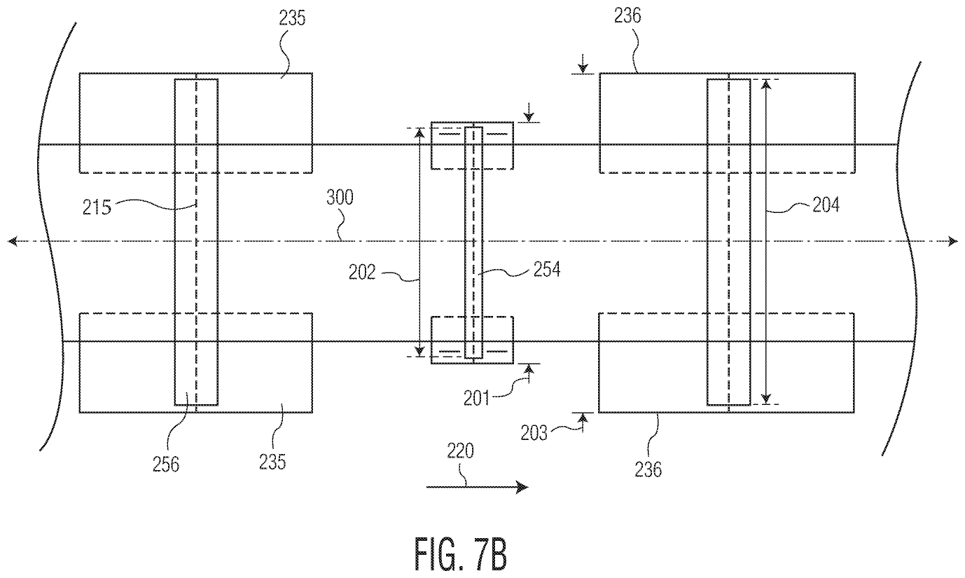

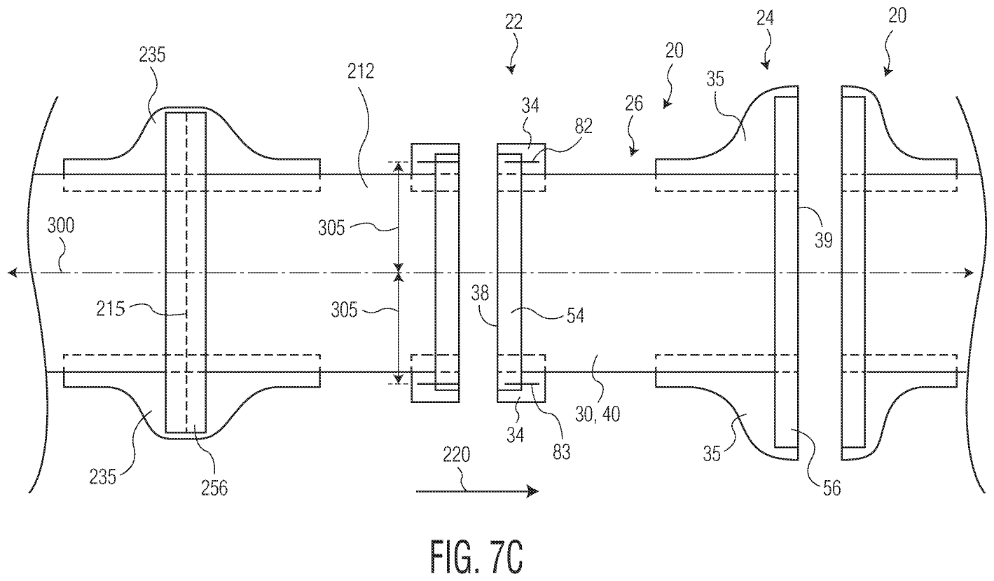

More particularly, FIGS. 7A-7C depicts one exemplary process 200 representing a portion of a longer process for forming absorbent articles 20 of the present disclosure from a continuous web of interconnected absorbent articles 20. Exemplary process 200 begins with moving first web 210, having side edges 247 and a central longitudinal axis 300, in the machine direction 220. First web 210 is shown with cut-lines 215 delineated, indicating locations along the first web 210 where the first web 210 will be severed to form individual absorbent articles 20. Cut-lines 215 does not represent any pre-cut or perforated portions of the web 210. Rather, cut-lines 215 are shown for illustration purposes only the different portions of the web 210 which will become different individual absorbent articles 20.

While moving the first web 210 in the machine direction 220, rear side panel material 235 are attached to the first web 210 adjacent side edges 247. Rear side panel material 235 may ultimately form rear side panels 35 of the absorbent article 20. Where the finished absorbent articles 20 include front side panels 34, front side panel materials 234 may also be attached to the first web 210 adjacent side edges 247 and form the front side panels 34 in the finished absorbent article 20.

As can be seen in FIGS. 7A-7C, the rear side panel material 235, and optionally the front side panel materials 234, may be attached to the first web 210 spanning cut-lines 215. Accordingly, when the web of interconnected absorbent articles of the process 200 is severed along cut-lines 215, the rear side panel material 235, and optionally the front side panel material 234, may be severed as well and form rear side panels 35, and optionally front side panels 34, of two separate absorbent articles 20. In this manner, the process 200 involves forming absorbent articles 20 in a front-to-front and rear-to-rear configuration, with a front region of a first absorbent article within the series of interconnected absorbent articles of the process 200 facing a front region of a second, adjacent and subsequent absorbent article. The second absorbent article has a rear region which faces a rear region of a third absorbent article which is adjacent to the second absorbent article and subsequent to the second absorbent article in the series of interconnected absorbent articles of the process 200.

In some embodiments, the first web 210 may comprise a bodyside liner material and an absorbent structure, with the rear side panel material 235, and optionally the front side panel material 234, may be attached to the bodyside liner material of the first web 210. Such components may be provided by suitable absorbent and/or web supply systems and methods as are known in the art. In such embodiments, an outer cover material may then be attached to the first web 210, forming second web 212, sandwiching the rear side panel material 235, and optionally the front side panel material 234, between the bodyside liner material and the outer cover material of the second web.

Although, it should be understood that many variations in these attachment steps are contemplated in different embodiments of the process 200. For example, in other embodiments, the first web 210 may comprise a bodyside liner material, an absorbent structure, and an outer cover material, and the rear side panel materials 235, and optionally the front side panel materials 234, may be attached to the outer cover material. In still further embodiments, the first web 210 may comprise a bodyside liner material and an absorbent structure, and the rear side panel material 235 may be coupled to the bodyside liner material. An outer cover material may then be coupled to the first web 210, sandwiching the rear side panel materials 235, and optionally the front side panel materials 234, between the bodyside liner material and the outer cover material. Of course, the order and attachment locations of the rear side panel material 235 and the front side panel material 234 may be different in other embodiments. For example, the front side panel materials 234 may be brought in and attached to the outer cover material after the outer cover material has been brought in an coupled to the web 210.

Once the outer cover material has been brought to the first web 210 to form the second web 212, a rear elastic waist material 256 may be coupled to the second web 212. In some embodiments, a front waist elastic material 254 may further be coupled to the second web 212. According to some embodiments, the rear elastic waist material 256, and the front waist elastic material 254 where present, may be coupled to the second web 212 spanning cut-lines 215. Accordingly, when the web of interconnected absorbent articles of the process 200 is severed along cut-lines 215, the rear elastic waist material 256, and the front waist elastic material 254 where present, may be severed as well and form rear waist elastic member 56 and optionally front waist elastic member 54 of two separate absorbent articles 20. Also, in this manner, the edges of the severed rear elastic waist material 256 and front waist elastic material 254, along with the edges of the severed web 212 (comprising a bodyside liner material and an outer cover material) may form the rear waist edge 39 and the front waist edge 38 of individual absorbent articles 20.

The rear elastic waist material 256 may be applied to the second web 212 at along a portion of the web 212 including rear side panel material 235 while the front waist elastic material 254 may be applied to the second web 212 along a portion of the web 212 including the front side panel material 234. The rear elastic waist material 256 may have a width 204 in a cross-machine direction that is perpendicular to the machine direction 220. The portion of the web 212 along which the rear elastic waist material 256 is applied may have a web width 203 measured between side edges 236. The values of the widths 204 and 203 may be similar to the widths 104 and 102, respectively, described previously related to the rear waist elastic member width and the article rear waist edge width. Similarly, the front elastic waist material 254 may have a width 202 and the portion of the web 212 along which the front elastic waist material 254 is applied may have a web width 201. The values of the widths 202 and 201 may be similar to the widths 103 and 101, respectively, described previously related to the front waist elastic member width and the article front waist edge width.

The rear elastic waist material 256 and the front waist elastic material 254, where present, may be applied to the second web 212 in an extended manner. For example, the waist materials 256 and optionally 254 may be applied at an extension of between about 0% and about 30%, in accordance with the extensions described previously for the waist elastic members 54 and 56 of article 20. Additionally, in various embodiments, the elastic waist materials 256, 254 may be applied at differing extensions, for example those described previously with respect to waist elastic members 54 and 56 of article 20.

It may be beneficial in some embodiments for the front and rear waist regions 22, 24 of individual absorbent articles 20 to comprise different stretch properties. Accordingly, where both the rear elastic waist material 256 and the front waist elastic material 254 are coupled to the second web 212, the elastic waist materials 256, 254 may be different materials having different elastomeric properties. For example, the elastic waist materials 256, 254 may be different materials according to the previously described embodiments with respect to waist elastic members 54 and 56 of article 20.

A fastening system may additionally be attached to the second web within the process 200. For instance, at least first fastening components (82 and 83) may be attached to the second web 212 after the front waist elastic material 254 has been applied, as shown in FIG. 7C. The first fastening components (82 and 83) may be applied to the second web so as to overlay either the front side panel material 234 or the outer cover material and to further overlay at least a portion of the front waist elastic material 254. The front waist elastic material 254 may be coupled to the second web 212 at a distance 305 from the longitudinal centerline 300 of the second web 212. The distance 305 may generally be between about 45 mm and about 90 mm. In further embodiments, the distances 305 may be any of the distances described with respect to distance 105.

As shown in FIGS. 7A-7C, the rear side panel material 235, and optionally the front side panel material 234 where included, may be applied as rectangular pieces of material. In such embodiments, after application of the rear elastic waist material 256 and optionally the front waist elastic material 254 to the second web 212, the rear side panel material 235 and/or the front side panel material 234 may be cut to form curved and/or angled portions so the rear side panel material 235 and/or the front side panel material 234 may better fit around the legs and waist of a wearer.

Finally, the continuous web of interconnected absorbent articles of the process 200 at this point may be severed along cut-lines 215 in order to form individual absorbent articles 20. As can be seen most clearly in FIG. 7C, severing the second web 212 along cut-lines 215 which are adjacent in the machine direction 220 results in severing the second web 212 along a first cut-line 215 which extends between front side panel material 234 and along a subsequent, adjacent cut-line 215 which extends between rear side panel material 235, and then along a subsequent, adjacent cut-line 215 which extends between front side panel material 234. In this manner, the process 200 results in individual absorbent articles 20 being separated from the second web 212 such that a rear waist region 24 of a first article 20 faces a rear waist region 24 of a second absorbent article 20, and wherein the front waist region 22 faces the front waist region 22 of a third absorbent article 20. This rear-to-rear and front-to-front facing configuration will continue to repeat itself throughout the process 20.

Once individual absorbent articles 20 are severed from the second web 212, the individual absorbent articles 20 may be folded and placed into a bag. In some embodiments, it may be desirable for all articles 20 within a bag to be oriented in the same direction. In such embodiments, the process 200 may further comprise a product rotator disposed prior to a folding mechanism and configured to rotate every other product. In such embodiments, all of the articles 20 may enter the folding mechanism in the same orientation and may thus be folded in the same manner. The folded articles 20 may then be placed into a bag where the articles 20 all have the same orientation.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

### EMBODIMENTS

Embodiment 1: An absorbent article with a front waist region having a front waist edge, a rear waist region having a rear waist edge, and a crotch region disposed between the front waist region and the rear waist region may comprise a chassis comprising a bodyside liner, an outer cover, and an absorbent body disposed between the bodyside liner and the outer cover, first and second elastomeric rear side panels, the first elastomeric rear side panel attached to a first longitudinally extending side edge of the chassis and the second elastomeric rear side panel attached to a second longitudinally extending side edge of the chassis, a portion of each of the first and second elastomeric rear side panels and the chassis forming the rear waist edge, first and second elastomeric front side panels, the first elastomeric front side panel attached to the first longitudinally extending side edge of the chassis and the second elastomeric front side panel attached to the second longitudinally extending side edge of the chassis, a portion of each of the first and second elastomeric front side panels and the chassis forming the front waist edge, and first and second mechanical fastening components coupled to the front waist region of the article and configured to releasably couple the front waist region to the rear waist region, wherein each of the rear waist edge and the front waist edge has a laterally extending width, and wherein a ratio of the rear waist edge width to the front waist edge width may be between about 1.5 and about 3.0.
Embodiment 2: The absorbent article of embodiment 1, wherein the ratio of the rear waist edge width to the front waist edge width may be between about 1.5 and about 2.0.
Embodiment 3: The absorbent article of any one of embodiments 1-2, wherein the first mechanical fastening component may be coupled to the first elastomeric front side panel and configured to releasably engage with an inner surface of the first elastomeric rear side panel and the second mechanical fastening component may coupled to the second elastomeric front side panel and configured to releasably engage with an inner surface of the second elastomeric rear side panel, and wherein inner edges of each of the first and second mechanical fastening components may be disposed between about 45 mm and about 90 mm from a central longitudinal axis of the article.
Embodiment 4: The absorbent article of embodiment 3, wherein the inner edges of each of the first and second mechanical fastening components may be disposed between about 65 mm and about 80 mm from the central longitudinal axis of the article.
Embodiment 5: The absorbent article of any one of embodiments 1-4, wherein inner edges of the first and second mechanical fastening components form an angle of between about 50 degrees and about 75 degrees with respect to the central longitudinal axis of the article, according to the Seam Position Test Method.
Embodiment 6: The absorbent article of any one of embodiments 1-5, further comprising a front elastomeric waistband coupled to the chassis and each of the first and second elastomeric front side panels, the front elastomeric waistband having a lateral width that may be between about 0 mm and about 10 mm less than the front waist edge width.
Embodiment 7: The absorbent article of embodiment 6, wherein at least a portion of each of the first mechanical fastening component and the second mechanical fastening component may be coupled to the front elastomeric waistband such that the front elastomeric waistband is disposed between the first mechanical fastening component and the second mechanical fastening component and the first elastomeric front side panel or the second elastomeric front side panel.
Embodiment 8: The absorbent article of any one of embodiments 1-7, further comprising a rear elastomeric waistband coupled to the chassis and each of the first and second elastomeric rear side panels, the rear elastomeric waistband having a lateral width that is between about 0 mm and about 10 mm less than the rear waist edge width.
Embodiment 9: The absorbent article of any one of embodiments 1-5, further comprising a front elastomeric waistband coupled to the chassis and each of the first and second elastomeric front side panels and a rear elastomeric waistband coupled to the chassis and each of the first and second elastomeric rear side panels, the front elastomeric waistband being coupled to the chassis and each of the first and second elastomeric front side panels at a first extension, and the a rear elastomeric waistband being coupled to the chassis and each of the first and second elastomeric rear side panels at a second extension, the first extension being different than the second extension.
Embodiment 10: An absorbent article with a front waist region having a front waist edge, a rear waist region having a rear waist edge, and a crotch region disposed between the front waist region and the rear waist region and having a body-facing surface and a garment-facing surface may comprise a chassis comprising a bodyside liner, an outer cover, and an absorbent body disposed between the bodyside liner and the outer cover, first and second elastomeric rear side panels, the first elastomeric rear side panel attached to a first longitudinally extending side edge of the chassis and the second elastomeric rear side panel attached to a second longitudinally extending side edge of the chassis, a portion of each of the first and second elastomeric rear side panels and the chassis forming the rear waist edge, first and second elastomeric front side panels, the first elastomeric front side panel attached to the first longitudinally extending side edge of the chassis and the second elastomeric front side panel attached to the second longitudinally extending side edge of the chassis, a portion of each of the first and second elastomeric front side panels and the chassis forming the front waist edge, first and second mechanical fastening components coupled to the front waist region of the article and configured to releasably couple to the front waist region to the rear waist region, and front and rear elastomeric waistbands, each of the front and rear elastomeric waistbands disposed on the garment-facing surface of the article and forming at least part of front waist edge and rear waist edge, respectively, the front elastomeric waistband having a lateral width that is between about 0 mm and about 10 mm less than the front waist edge width, and the rear elastomeric waistband having a lateral width that is between about 0 mm and about 10 mm less than the rear waist edge width. Each of the rear waist edge and the front waist edge has a laterally extending width, and wherein a ratio of the rear waist edge width to the front waist edge width may be greater than about 1.5.
Embodiment 11: The absorbent article of embodiment 10, wherein the front elastomeric waistband may be coupled to the chassis and each of the first and second elastomeric front side panels at a first extension, and the a rear elastomeric waistband may be coupled to the chassis and each of the first and second elastomeric rear side panels at a second extension, and wherein the first extension is different than the second extension.
Embodiment 12: The absorbent article of embodiment 11, wherein the first extension may be between about 0% and about 30%, and wherein the second extension is greater than the first extension.
Embodiment 13: The absorbent article of any one of the embodiments 10-12, wherein the front side panels have an extensibility that is less than the extensibility of the rear side panels.
Embodiment 14: The absorbent article of any one of embodiments 10-13, wherein inner edges of each of the first and second mechanical fastening components may be disposed between about 45 mm and about 90 mm from a central longitudinal axis of the article.
Embodiment 15: The absorbent article of any one of embodiments 10-14, wherein a ratio of the rear waist edge width to the front waist edge width is between about 1.5 and about 3.0.
Embodiment 16: The absorbent article of any one of claims 10-15, wherein each of the first and second mechanical fastening components forms an angle of between about 5 degrees and about 30 degrees with respect to a central longitudinal axis of the article.
Embodiment 17: An absorbent article with a front waist region having a front waist edge, a rear waist region having a rear waist edge, and a crotch region disposed between the front waist region and the rear waist region and having a body-facing surface and a garment-facing surface may comprise a chassis comprising a bodyside liner, an outer cover, and an absorbent body disposed between the bodyside liner and the outer cover, first and second elastomeric rear side panels, the first elastomeric rear side panel attached to a first longitudinally extending side edge of the chassis and the second elastomeric rear side panel attached to a second longitudinally extending side edge of the chassis, a portion of each of the first and second elastomeric rear side panels and the chassis forming the rear waist edge, first and second elastomeric front side panels, the first elastomeric front side panel attached to the first longitudinally extending side edge of the chassis and the second elastomeric front side panel attached to the second longitudinally extending side edge of the chassis, a portion of each of the first and second elastomeric front side panels and the chassis forming the front waist edge, front and rear elastomeric waistbands, each of the front and rear elastomeric waistbands disposed on the garment-facing surface of the article, the front elastomeric waistband having a lateral width that is between about 0 mm and about 10 mm less than the front waist edge width and the rear elastomeric waistband having a lateral width that is between about 0 mm and about 10 mm less than the rear waist edge width, and first and second mechanical fastening components coupled to the front waist region of the article and configured to releasably couple to the front waist region to the rear waist region, at least a portion of the first and second mechanical fastening components are directly coupled to the front elastomeric waistband. Each of the rear waist edge and the front waist edge has a laterally extending width, and wherein a ratio of the rear waist edge width and the front waist edge width may be greater than about 1.5.
Embodiment 18: The absorbent article of embodiment 17, wherein a first surface of the front elastomeric waistband may be coupled to the first and second elastomeric front side panels and a second surface of the front elastomeric waistband may be coupled to the first and second mechanical fastening components.
Embodiment 19: The absorbent article of any one of embodiments 17-18, wherein the first mechanical fastening component may be coupled to the first elastomeric front side panel and the second mechanical fastening component may be coupled to the second elastomeric front side panel, the first and second mechanical fastening components each having inner edges which may be disposed between about 45 mm and about 90 mm from a central longitudinal axis of the article.
Embodiment 20: The absorbent article of any one of embodiments 17-19, wherein each of the first and second mechanical fastening components may form a fastener angle of between about 5.0 degrees and about 30 degrees with respect to a central longitudinal axis of the article.
Embodiment 21: The absorbent article of embodiment 20, wherein an outer edge of the first elastomeric rear side panel may form an angle with respect to the central longitudinal axis that is within about 10 degrees of the fastener angle formed by the first mechanical fastening component and the central longitudinal axis, and wherein an outer edge of the second elastomeric rear side panel may form an angle with respect to the central longitudinal axis that is within about 10 degrees of the fastener angle formed by the second mechanical fastening component and the central longitudinal axis.
Embodiment 22: An absorbent article with a front waist region having a front waist edge, a rear waist region having a rear waist edge, and a crotch region disposed between the front waist region and the rear waist region may comprise a chassis comprising a bodyside liner, an outer cover, and an absorbent body disposed between the bodyside liner and the outer cover, first and second elastomeric rear side panels, the first elastomeric rear side panel attached to a first longitudinally extending side edge of the chassis and the second elastomeric rear side panel attached to a second longitudinally extending side edge of the chassis, a portion of each of the first and second elastomeric rear side panels and the chassis forming the rear waist edge, and the chassis forming the front waist edge, first and second mechanical fastening components coupled to the front waist region of the article and configured to releasably couple the front waist region to the rear waist region, wherein inner edges of the first and second fastening components form an angle of between about 50 degrees and about 75 degrees with respect to the central longitudinal axis of the article, according to the Seam Position Test Method.
Embodiment 23: The absorbent article of embodiment 22, wherein inner edges of the first and second fastening components form an angle of between about 55 degrees and about 70 degrees with respect to the central longitudinal axis of the article, according to the Seam Position Test Method.
Embodiment 24: The absorbent article of embodiment 22, wherein inner edges of the first and second fastening components form an angle of between about 50 degrees and about 65 degrees with respect to the central longitudinal axis of the article, according to the Seam Position Test Method.
Embodiment 25: The absorbent article of any one of embodiments 22-24, wherein the ratio of the rear waist edge width to the front waist edge width may be between about 1.5 and about 2.0.
Embodiment 26: The absorbent article of any one of embodiments 22-25, wherein the first mechanical fastening component may be coupled to the first elastomeric front side panel and configured to releasably engage with an inner surface of the first elastomeric rear side panel and the second mechanical fastening component may coupled to the second elastomeric front side panel and configured to releasably engage with an inner surface of the second elastomeric rear side panel, and wherein inner edges of each of the first and second mechanical fastening components may be disposed between about 45 mm and about 90 mm from a central longitudinal axis of the article.
Embodiment 27: The absorbent article of embodiment 26, wherein the inner edges of each of the first and second mechanical fastening components may be disposed between about 65 mm and about 80 mm from the central longitudinal axis of the article.
Embodiment 28: The absorbent article of any one of embodiments 22-27, further comprising a front elastomeric waistband coupled to the chassis and each of the first and second elastomeric front side panels, the front elastomeric waistband having a lateral width that may be between about 0 mm and about 10 mm less than the front waist edge width.
Embodiment 29: The absorbent article of embodiment 28, wherein at least a portion of each of the first mechanical fastening component and the second mechanical fastening component may be coupled to the front elastomeric waistband such that the front elastomeric waistband is disposed between the first mechanical fastening component and the second mechanical fastening component and the first elastomeric front side panel or the second elastomeric front side panel.
Embodiment 30: The absorbent article of any one of embodiments 22-29, further comprising a rear elastomeric waistband coupled to the chassis and each of the first and second elastomeric rear side panels, the rear elastomeric waistband having a lateral width that is between about 0 mm and about 10 mm less than the rear waist edge width.
Embodiment 31: The absorbent article of any one of embodiments 22-27, further comprising a front elastomeric waistband coupled to the chassis and each of the first and second elastomeric front side panels and a rear elastomeric waistband coupled to the chassis and each of the first and second elastomeric rear side panels, the front elastomeric waistband being coupled to the chassis and each of the first and second elastomeric front side panels at a first extension, and the a rear elastomeric waistband being coupled to the chassis and each of the first and second elastomeric rear side panels at a second extension, the first extension different than the second extension.

What is claimed is:

1. An absorbent article with a front waist region having a front waist edge, a rear waist region having a rear waist edge, and a crotch region disposed between the front waist region and the rear waist region, the article comprising:
a chassis comprising a bodyside liner, an outer cover, and an absorbent body disposed between the bodyside liner and the outer cover;
first and second elastomeric rear side panels, the first elastomeric rear side panel attached to a first longitudinally extending side edge of the chassis and the second elastomeric rear side panel attached to a second longitudinally extending side edge of the chassis, a portion of each of the first and second elastomeric rear side panels and the chassis forming the rear waist edge;
first and second elastomeric front side panels, the first elastomeric front side panel attached to the first longitudinally extending side edge of the chassis and the second elastomeric front side panel attached to the second longitudinally extending side edge of the chassis, a portion of each of the first and second elastomeric front side panels and the chassis forming the front waist edge; and
first and second mechanical fastening components coupled to the front waist region of the article and configured to releasably couple the front waist region to the rear waist region;
wherein each of the rear waist edge and the front waist edge has a laterally extending width, and wherein a ratio of the rear waist edge width to the front waist edge width is between about 1.5 and about 3.0.

2. The absorbent article of claim 1, wherein the ratio of the rear waist edge width to the front waist edge width is between about 1.5 and about 2.0.

3. The absorbent article of claim 1, wherein the first mechanical fastening component is coupled to the first elastomeric front side panel and configured to releasably engage with an inner surface of the first elastomeric rear side panel and the second mechanical fastening component is coupled to the second elastomeric front side panel and configured to releasably engage with an inner surface of the second elastomeric rear side panel, and wherein inner edges of each of the first and second mechanical fastening components are disposed between about 45 mm and about 90 mm from a central longitudinal axis of the article.

4. The absorbent article of claim 1, wherein inner edges of the first and second mechanical fastening components form an angle of between about 50 degrees and about 75 degrees with respect to the central longitudinal axis of the article, according to the Seam Position Test Method.

5. The absorbent article of claim 1, further comprising a front elastomeric waistband coupled to the chassis and each of the first and second elastomeric front side panels, the front elastomeric waistband having a lateral width that is between about 0 mm and about 10 mm less than the front waist edge width.

6. The absorbent article of claim 5, wherein at least a portion of each of the first mechanical fastening component and the second mechanical fastening component is coupled to the front elastomeric waistband such that the front elastomeric waistband is disposed between the first mechanical fastening component and the second mechanical fastening component and the first elastomeric front side panel or the second elastomeric front side panel.

7. The absorbent article of claim 1, further comprising a rear elastomeric waistband coupled to the chassis and each of the first and second elastomeric rear side panels, the rear elastomeric waistband having a lateral width that is between about 0 mm and about 10 mm less than the rear waist edge width.

8. The absorbent article of claim 1, further comprising a front elastomeric waistband coupled to the chassis and each of the first and second elastomeric front side panels and a rear elastomeric waistband coupled to the chassis and each of the first and second elastomeric rear side panels, the front elastomeric waistband being coupled to the chassis and each of the first and second elastomeric front side panels at a first extension, and the a rear elastomeric waistband being coupled to the chassis and each of the first and second elastomeric rear side panels at a second extension, the first extension being different than the second extension.

9. An absorbent article with a front waist region having a front waist edge, a rear waist region having a rear waist edge, and a crotch region disposed between the front waist region and the rear waist region and having a body-facing surface and a garment-facing surface, the article comprising:
a chassis comprising a bodyside liner, an outer cover, and an absorbent body disposed between the bodyside liner and the outer cover;
first and second elastomeric rear side panels, the first elastomeric rear side panel attached to a first longitudinally extending side edge of the chassis and the second elastomeric rear side panel attached to a second longitudinally extending side edge of the chassis, a portion of each of the first and second elastomeric rear side panels and the chassis forming the rear waist edge;
first and second elastomeric front side panels, the first elastomeric front side panel attached to the first longitudinally extending side edge of the chassis and the second elastomeric front side panel attached to the second longitudinally extending side edge of the chassis, a portion of each of the first and second elastomeric front side panels and the chassis forming the front waist edge;
first and second mechanical fastening components coupled to the front waist region of the article and configured to releasably couple to the front waist region to the rear waist region; and
front and rear elastomeric waistbands, each of the front and rear elastomeric waistbands disposed on the garment-facing surface of the article and forming at least part of front waist edge and rear waist edge, respectively, the front elastomeric waistband having a lateral width that is between about 0 mm and about 10 mm less than the front waist edge width, and the rear elastomeric waistband having a lateral width that is between about 0 mm and about 10 mm less than the rear waist edge width, and
wherein each of the rear waist edge and the front waist edge has a laterally extending width, and wherein a ratio of the rear waist edge width to the front waist edge width is greater than about 1.5.

10. The absorbent article of claim 9, wherein the front elastomeric waistband is coupled to the chassis and each of the first and second elastomeric front side panels at a first extension, and the a rear elastomeric waistband is coupled to the chassis and each of the first and second elastomeric rear side panels at a second extension, and wherein the first extension is different than the second extension.

11. The absorbent article of claim 10, wherein the first extension is between about 0% and about 30%, and wherein the second extension is greater than the first extension.

12. The absorbent article of claim 9, wherein the front side panels have an extensibility that is less than the extensibility of the rear side panels.

13. The absorbent article of claim 9, wherein inner edges of each of the first and second mechanical fastening components are disposed between about 45 mm and about 90 mm from a central longitudinal axis of the article.

14. The absorbent article of claim 9, wherein a ratio of the rear waist edge width to the front waist edge width is between about 1.5 and about 3.0.

15. The absorbent article of claim 9, wherein each of the first and second mechanical fastening components forms an angle of between about 5 degrees and about 30 degrees with respect to a central longitudinal axis of the article.

16. An absorbent article with a front waist region having a front waist edge, a rear waist region having a rear waist edge, and a crotch region disposed between the front waist region and the rear waist region and having a body-facing surface and a garment-facing surface, the article comprising:
a chassis comprising a bodyside liner, an outer cover, and an absorbent body disposed between the bodyside liner and the outer cover;
first and second elastomeric rear side panels, the first elastomeric rear side panel attached to a first longitudinally extending side edge of the chassis and the second elastomeric rear side panel attached to a second longitudinally extending side edge of the chassis, a portion of each of the first and second elastomeric rear side panels and the chassis forming the rear waist edge;
first and second elastomeric front side panels, the first elastomeric front side panel attached to the first longitudinally extending side edge of the chassis and the second elastomeric front side panel attached to the second longitudinally extending side edge of the chassis, a portion of each of the first and second elastomeric front side panels and the chassis forming the front waist edge;
front and rear elastomeric waistbands, each of the front and rear elastomeric waistbands disposed on the garment-facing surface of the article, the front elastomeric waistband having a lateral width that is between about 0 mm and about 10 mm less than the front waist edge width and the rear elastomeric waistband having a lateral width that is between about 0 mm and about 10 mm less than the rear waist edge width, and
first and second mechanical fastening components coupled to the front waist region of the article and configured to releasably couple to the front waist region to the rear waist region, at least a portion of the first and second mechanical fastening components are directly coupled to the front elastomeric waistband; and
wherein each of the rear waist edge and the front waist edge has a laterally extending width, and wherein a ratio of the rear waist edge width and the front waist edge width is greater than about 1.5.

17. The absorbent article of claim 16, wherein a first surface of the front elastomeric waistband is coupled to the first and second elastomeric front side panels and a second surface of the front elastomeric waistband is coupled to the first and second mechanical fastening components.

18. The absorbent article of claim 16, wherein the first mechanical fastening component is coupled to the first elastomeric front side panel and the second mechanical fastening component is coupled to the second elastomeric front side panel, the first and second mechanical fastening components each having inner edges which are disposed between about 45 mm and about 90 mm from a central longitudinal axis of the article.

19. The absorbent article of claim 16, wherein each of the first and second mechanical fastening components forms a fastener angle of between about 5.0 degrees and about 30 degrees with respect to a central longitudinal axis of the article.

20. The absorbent article of claim 19, wherein an outer edge of the first elastomeric rear side panel forms an angle with respect to the central longitudinal axis that is within about 10 degrees of the fastener angle formed by the first mechanical fastening component and the central longitudinal axis, and wherein an outer edge of the second elastomeric rear side panel forms an angle with respect to the central longitudinal axis that is within about 10 degrees of the fastener angle formed by the second mechanical fastening component and the central longitudinal axis.
